SEQUENCE LISTING (10 sequences)

SEQ ID NO: 1 - protein, 602 aa, Nipah henipavirus
MPTESKKVRFENTASDKGKNPSKVIKSYYGTMDIKKINEGLLDSKILSAFNTVIALLGSIVIIVMNIMIIQNYTRSTDNQAMIKDALQSIQQQIKGLADKIGTEIGPKVSLIDTSSTITIPANIGLLGSKISQSTASINENVNEKCKFTLPPLKIHECNISCPNPLPFREYKPQTEGVSNLVGLPNNICLQKTSNQILKPKLISYTLPVVGQSGTCITDPLLAMDEGYFAYSHLEKIGSCSRGVSKQRIIGVGEVLDRGDEVPSLFMTNVWTPSNPNTVYHCSAVYNNEFYYVLCAVSVVGDPILNSTYWSGSLMMTRLAVKPKNNGESYNQHQFALRNIEKGKYDKVMPYGPSGIKQGDTLYFPAVGFLVRTEFTYNDSNCPIAECQYSKPENCRLSMGIRPNSHYILRSGLLKYNLSDEENSKIVFIEISDQRLSIGSPSKIYDSLGQPVFYQASFSWDTMIKFGDVQTVNPLVVNWRDNTVISRPGQSQCPRFNKCPEVCWEGVYNDAFLIDRINWISAGVFLDSNQTAENPVFTVFKDNEVLYRAQLASEDTNAQKTITNCFLLKNKIWCISLVEIYDTGDNVIRPKLFAVKIPEQCT

SEQ ID NO: 2 - protein, 569 aa, synthetic construct
MKKINEGLLDSKILSAFNTVIALLGSIVIIVMNIMIIQNYTRSTDNQAMIKDALQSIQQQIKGLADKIGTEIGPKVSLIDTSSTITIPANIGLLGSKISQSTASINENVNEKCKFTLPPLKIHECNISCPNPLPFREYKPQTEGVSNLVGLPNNICLQKTSNQILKPKLISYTLPVVGQSGTCITDPLLAMDEGYFAYSHLEKIGSCSRGVSKQRIIGVGEVLDRGDEVPSLFMTNVWTPSNPNTVYHCSAVYNNEFYYVLCAVSVVGDPILNSTYWSGSLMMTRLAVKPKNNGESYNQHQFALRNIEKGKYDKVMPYGPSGIKQGDTLYFPAVGFLVRTEFTYNDSNCPIAECQYSKPENCRLSMGIRPNSHYILRSGLLKYNLSDEENSKIVFIEISDQRLSIGSPSKIYDSLGQPVFYQASFSWDTMIKFGDVQTVNPLVVNWRDNTVISRPGQSQCPRFNKCPEVCWEGVYNDAFLIDRINWISAGVFLDSNQTAENPVFTVFKDNEVLYRAQLASEDTNAQKTITNCFLLKNKIWCISLVEIYDTGDNVIRPKLFAVKIPEQCT

SEQ ID NO: 3 - protein, 546 aa, Nipah henipavirus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 4 - protein, 524 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 5 - protein, 520 aa, synthetic construct
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYLIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPILLERLDVGTNLGNAIAKLEDAKELLESSDQILRSMKGLSSTCIVYILIAVCLGGLIGIPALICCCRGR

SEQ ID NO: 6 - protein, 601 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGEDCHAPTYLPAEVDGDVKLSSNLVILPGQDLQYVLATYDTSAVEHAVVYYVYSPSRLSSYFYPFRLPIKGVPIELQVECFTWDQKLWCRHFCVLADSESGGHITHSGMVGMGVSCTVTREDGTNRRGG

SEQ ID NO: 7 - protein, 842 aa, synthetic construct
MKKINEGLLDSKILSAFNTVIALLGSIVIIVMNIMIIQNYTRSTDNQAMIKDALQSIQQQIKGLADKIGTEIGPKVSLIDTSSTITIPANIGLLGSKISQSTASINENVNEKCKFTLPPLKIHECNISCPNPLPFREYKPQTEGVSNLVGLPNNICLQKTSNQILKPKLISYTLPVVGQSGTCITDPLLAMDEGYFAYSHLEKIGSCSRGVSKQRIIGVGEVLDRGDEVPSLFMTNVWTPSNPNTVYHCSAVYNNEFYYVLCAVSVVGDPILNSTYWSGSLMMTRLAVKPKNNGESYNQHQFALRNIEKGKYDKVMPYGPSGIKQGDTLYFPAVGFLVRTEFTYNDSNCPIAECQYSKPENCRLSMGIRPNSHYILRSGLLKYNLSDEENSKIVFIEISDQRLSIGSPSKIYDSLGQPVFYQASFSWDTMIKFGDVQTVNPLVVNWRDNTVISRPGQSQCPRFNKCPVVCAEGVYNDAFLIDRINWISAGVFLDSNATAANPVFTVFKDNEVLYRAQLASEDTNAQKTITNCFLLKNKIWCISLVEIYDTGDNVIRPKLFAVKIPEQCTDIGGGGSGGGGSGGGGSGGGGSDIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKRGGGGSGGGGSGGGGSGGGGSQVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSS

SEQ ID NO: 8 - protein, 5 aa, synthetic construct
  REPEAT 1..5: note = Sequence may be repeated n times where n is 1, 2, 3, 4, or 5
GGGGS

SEQ ID NO: 9 - protein, 5 aa, synthetic construct
  REPEAT 1..5: note = Sequence may be repeated n times where n is 1, 2, 3, 4, or 5
GGGGA

SEQ ID NO: 10 - protein, 4 aa, synthetic construct
AAAA

(19) United States
(12) Patent Application Publication
(10) Pub. No.: US 2025/0051797 A1
(43) Pub. Date: Feb. 13, 2025

(54) ENGINEERED VIRAL PARTICLES AND USES OF THE SAME

(71) Applicant: INTERIUS BIOTHERAPEUTICS, INC., Philadelphia, PA (US)

(72) Inventors: James I. Andorko, Philadelphia, PA (US); Mohit Sachdeva, Philadelphia, PA (US)

(21) Appl. No.: 18/718,667

(22) PCT Filed: Dec. 12, 2022
(86) PCT No.: PCT/US2022/081332

§ 371 Date: Jun. 11, 2024

Related U.S. Application Data
(60) Provisional application No. 63/265,351, filed on Dec. 13, 2021.

(51) Int. Cl. C12N 15/86; A61K 35/76; A61K 48/00

(52) U.S. Cl. CPC C12N 15/86; A61K 35/76; A61K 48/0041; C12N 2740/15023; C12N 2740/15033; C12N 2740/15043; C12N 2740/15045; C12N 2760/18222; C12N 2760/18422

(57) ABSTRACT

Provided for herein are compositions and methods that can be used to transduce cells and deliver genetic information that can then be expressed in the transduced cell. Also provided herein are methods of treating a disease in a subject using the compositions and methods provided.

### CROSS REFERENCE TO RELATE APPLICATIONS

This application claims the benefit of U.S. Provisional Application Ser. No. 63/265,351 filed 13 Dec. 2021, which is hereby incorporated by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Nov. 18, 2022, is named “148165.001002_SL.xml” and is 14.3 kilobytes in size.

### BACKGROUND

Lentiviruses are common vectors used in gene therapy because they can transduce non-dividing cells and offer stable integration into a target cell's genome. The host range of lentivirus vectors can be altered by pseudotyping with glycoproteins derived from other viruses. Such pseudotyped lentiviral vectors then exhibit a receptor phenotype similar to the virus from which the envelope protein was derived. Depending on the host range of said virus, the pseudotyped retroviral vectors will then have a broadened or narrowed host range as compared to vector particles having the incorporated homologous retroviral envelope proteins. There is still a need to overcome the shortcomings of certain pseudotyped viruses. The embodiments provided herein fulfill these needs as well as others.

### BRIEF SUMMARY

In some embodiments, the disclosure provides for pseudotyped viral-like particles or viral vectors. In some embodiments, pseudotyped viral-like particles or viral vectors comprise a targeting polypeptide comprising a heterologous targeting moiety linked to a protein derived from an envelope glycoprotein G or H of a virus of the Paramyxoviridae family; a truncated protein derived from an envelope glycoprotein G or H of a virus of the Paramyxoviridae family, wherein the truncated protein does not comprise (e.g., free of) a targeting moiety; a glycoprotein derived from an envelope glycoprotein F of a virus of the Paramyxoviridae family, wherein the targeting polypeptide and the truncated protein comprise the same or different envelope glycoproteins.

In some embodiments, pseudotyped viral-like particles or viral vectors comprise a plurality of polypeptides comprising the general amino acid formula of:

[PG]-[RR]-Linker-[TM];

[EG]; and

[GF], wherein:
PG is an envelope glycoprotein G or H polypeptide of a virus of the Paramyxoviridae family, such as, but not limited to as provided herein; RR is an optional restriction site sequence, such as, but not limited to, as provided herein; Linker is peptide linker such as, but not limited to, those provided herein; TM is a targeting moiety; EG is an envelope glycoprotein G or H of a virus of the Paramyxoviridae family, such as, but not limited to, as provided herein; and GF is an envelope glycoprotein F of a virus of the Paramyxoviridae family, such as, but not limited to, as provided herein.

In some embodiments, pharmaceutical compositions comprising the pseudotyped viral-like particles or viral vectors provided herein are provided.

In some embodiments, methods of delivering a cargo of interest to a cell are provided. In some embodiments, the method comprises contacting the cell with the pseudotyped viral-like particle or viral vector provided herein.

In some embodiments, methods of delivering a cargo of interest to a cell in a subject are provided. In some embodiments, the method comprises administering to the subject the pseudotyped viral-like particle or viral vector provided herein.

In some embodiments, methods for of delivering a chimeric antigen receptor to a T-cell in a subject are provided. In some embodiments, the method comprises administering to the subject the pseudotyped viral-like particle or viral vector provided herein, or a pharmaceutical composition comprising the same, wherein the pseudotyped viral-like particle or viral vector comprises a heterologous nucleic acid molecule encoding the chimeric antigen receptor.

In some embodiments, methods of treating cancer in a subject are provided. In some embodiments, the method comprises administering to the subject the pseudotyped viral-like particle or viral vector provided herein, or a pharmaceutical composition comprising the same, wherein the pseudotyped viral-like particle or viral vector comprises a heterologous nucleic acid molecule encoding the chimeric antigen receptor.

In some embodiments, nucleic acid molecules encoding a targeting polypeptide comprising a heterologous targeting moiety linked to a protein derived from an envelope glycoprotein G or H of a virus of the Paramyxoviridae family; a truncated protein derived from an envelope glycoprotein G or H of a virus of the Paramyxoviridae family, wherein the truncated protein does not comprise (e.g., free of) a targeting moiety; a glycoprotein derived from an envelope glycoprotein F of a virus of the Paramyxoviridae family, wherein the targeting polypeptide and the truncated protein comprise the same or different envelope glycoproteins are provided.

In some embodiments, plurality of nucleic acid molecules encoding a targeting polypeptide comprising a heterologous targeting moiety linked to a protein derived from an envelope glycoprotein G or H of a virus of the Paramyxoviridae family; a truncated protein derived from an envelope glycoprotein G or H of a virus of the Paramyxoviridae family, wherein the truncated protein does not comprise (e.g., free of) a targeting moiety; a glycoprotein derived from an envelope glycoprotein F of a virus of the Paramyxoviridae family, wherein the targeting polypeptide and the truncated protein comprise the same or different envelope glycoproteins is provided.

In some embodiments, methods of making a pseudotyped viral-like particle or viral vector provided herein are provided. In some embodiments, kits comprising a pharmaceutical composition comprising the pseudotyped viral-like particle or viral vector provided herein are provided.

### BRIEF DESCRIPTION OF THE FIGURES

FIG. 1 illustrates embodiments provided for herein.

FIG. 2 illustrates GFP transduction in cells with viral constructs as provided for herein. FIG. 2A illustrates the transduction results for a NiV-G comprising a CD8 DARPin targeting moiety+/−an untargeted NiV-G. FIG. 2B illustrates the transduction results for a NiV-G comprising a CD7 scFv targeting moiety+/−an untargeted NiV-G. FIG. 2C illustrates the transduction results for a NiV-G comprising a CD7 VHH targeting moiety+/−an untargeted NiV-G. FIG. 2D illustrates the transduction results for a NiV-G comprising a human CD7 scFv targeting moiety+/−an untargeted NiV-G. FIG. 2E illustrates the control no binder condition.

FIG. 3 illustrates CAR20 transduction in cells with viral constructs as provided for herein. FIG. 3A illustrates transduction with viral constructs comprising a targeted NiV-G alone, an untargeted NiV-G alone, or a combination of targeted and untargeted NiV-G wherein a 41BB co-stimulatory domain is also present. FIG. 3B illustrates transduction with viral constructs comprising a targeted NiV-G alone, an untargeted NiV-G alone, or a combination of targeted and untargeted NiV-G wherein no additional co-stimulatory domain is present.

### DETAILED DESCRIPTION

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, ±5%, ±1%, or ±0.1% from the specified value, as such variations are appropriate to perform the disclosed methods.

“Activation,” as used herein in reference to a T cell, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. The term “antigen” can also refer to a molecule that an antibody or antibody-like molecule can bind to or is recognized by the antibody or antibody-like molecule.

The term “antibody molecule,” “antibody” or antigen binding domain, as that term is used herein, refers to a polypeptide, e.g., an immunoglobulin chain or fragment thereof, comprising at least one functional immunoglobulin variable domain sequence. An antibody molecule encompasses antibodies (e.g., full-length antibodies) and antibody fragments. In some embodiments, an antibody molecule comprises an antigen binding or functional fragment of a full-length antibody, or a full-length immunoglobulin chain. For example, a full-length antibody is an immunoglobulin (Ig) molecule (e.g., an IgG antibody) that is naturally occurring or formed by normal immunoglobulin gene fragment recombinatorial processes. In embodiments, an antibody molecule refers to an immunologically active, antigen binding portion of an immunoglobulin molecule, such as an antibody fragment. An antibody fragment, e.g., functional fragment, comprises a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full-length antibodies and antibody fragments, e.g., dAb (domain antibody), single chain, Fab, Fab′, and F(ab′)2 fragments, and single chain variable fragments (scFvs).

The term “antibody molecule” also encompasses whole or antigen binding fragments of domain, or single domain, antibodies, which can also be referred to as “sdAb” or “VHH.” Domain antibodies comprise either VH or VL that can act as stand-alone, antibody fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. (“Sequence of proteins of immunological interest,” US Public Health Services, NIH Bethesda, MD, Publication No. 91, which is hereby incorporated by reference). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113. Domain antibodies are also described in WO2004041862 and WO2016065323, each of which is hereby incorporated by reference. The domain antibodies can be a targeting moiety as described herein.

Antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, or hexavalent), or with higher orders of specificity (e.g, tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material, such as a cell, derived from a subject to which it is later to be re-introduced into the same subject.

As used herein, the term “allogeneic” is meant to refer to material, such as a cell, derived from one subject that is later introduced into a different subject.

As used herein, the term “cargo” is meant to refer to any product that may be encoded by a nucleic acid molecule. As non-limiting examples, “cargo” may refer to an siRNA, an shRNA, a peptide, a polypeptide, a protein, a viral payload, a viral genome, or a combination thereof. In some embodiments, the polypeptide is a chimeric antigen receptor (“CAR”).

A “chimeric antigen receptor” or “CAR” as used herein refers to an antigen-binding domain that is fused to an intracellular signaling domain capable of activating or stimulating an immune cell. Most commonly, the CAR's extracellular binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). In various embodiments, this scFv is fused to a transmembrane domain and then to an intracellular signaling domain. However, the antigen binding domain can be any molecule that can bind to the to target on the cell. For example, the antigen binding domain of a CAR can be an antibody, a scFv antibody, an antigen binding domain, an ankyrin repeat (e.g. DARPIN), a VHH domain antibody, a nanobody, single domain antibody, a FN3 domain, or any combination thereof. In some embodiments, a CAR includes those that solely provide CD3ζ signals upon antigen binding. In some embodiments, the CAR includes those that provide both costimulation (e.g. CD28 or CD137) and activation (CD3 ζ). In some embodiments, the CARs include those that provide multiple costimulation (e.g. CD28 and CD137) and activation (CD3.zeta.). In various embodiments, the CAR is selected to have high affinity or avidity for the antigen. In some embodiments, the CAR comprises the 4-1BB domain as well. These are merely illustrative in nature and are not limiting to the present embodiments and any chimeric antigen receptor can be delivered in conjunction with the viral particles and vectors provided for herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of” or “consists.”

As used herein, the term “contacting” means bringing together of two elements in an in vitro system or an in vivo system. For example, “contacting” a vector with a cell or with an individual or patient or cell includes the administration of the vector to an individual or patient, such as a human, as well as, for example, introducing a compound into a sample containing a cellular or purified preparation containing the cell.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune cell activation compared to the immune cell activation detected in the absence of the composition. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. In some embodiments, the epitope is about 4-18 amino acids, about 5-16 amino acids, about 6-14 amino acids, about 7-12, or about 8-10 amino acids. One skilled in the art understands that generally the overall three-dimensional structure, rather than the specific linear sequence of the molecule, is the main criterion of antigenic specificity and, therefore, distinguishes one epitope from another. Based on the present disclosure, a peptide can be an epitope.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the phrase “ex vivo” in reference to a cell being transduced, transfected or transformed ex vivo, refers to a cell being transduced, transfected or transformed outside of the subject, that is with the cells being removed from the subject before such cells are transduced, transfected or transformed.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another. In some embodiments, the various domains or proteins provided for herein are linked or fused directly to one another or a linker sequences, such as a glycine/serine sequence link the two domains together.

“Identity” as used herein refers to the subunit sequence identity between two polymeric molecules such as between two nucleic acid or amino acid molecules, such as, between two polynucleotide or polypeptide molecules. When two amino acid sequences have the same residues at the same positions; e.g., if a position in each of two polypeptide molecules is occupied by an Arginine, then they are identical at that position. The identity or extent to which two amino acid or two nucleic acid sequences have the same residues at the same positions in an alignment is often expressed as a percentage. The identity between two amino acid or two nucleic acid sequences is a direct function of the number of matching or identical positions; e.g., if half of the positions in two sequences are identical, the two sequences are 50% identical; if 90% of the positions (e.g., 9 of 10), are matched or identical, the two amino acids sequences are 90% identical.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity can be measured/determined using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e³ and e¹⁰⁰ indicating a closely related sequence. In some embodiments, sequence identity is determined by using BLAST with the default settings.

To the extent embodiments provided for herein, includes composition comprising various proteins, these proteins may, in some instances, comprise amino acid sequences that have sequence identity to the amino acid sequences disclosed herein. Therefore, in certain embodiments, depending on the particular sequence, the degree of sequence identity is preferably greater than 50% (e.g. 60%, 70%, 75%, 80%, 85%, 90%, 91%, 92%, 93%, 94%, 95%, 96%, 97%, 98%, 99% or more) to the SEQ ID NOs disclosed herein. These proteins may include homologs, orthologues, allelic variants and functional mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty−12 and gap extension penalty=1.

These proteins may, compared to the disclosed proteins, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, Substitution of single amino acids within these families does not have a major effect on the biological activity. The proteins may have one or more (e.g. 1, 2, 3, 4, 5, 6, 7, 8, 9, 10, etc.) single amino acid deletions relative to the disclosed protein sequences. The proteins may also include one or more (e.g. 1, 2, 3, 4, 5, 6, 7, 8, 9, 10, etc.) insertions (e.g. each of 1, 2, 3, 4 or 5 amino acids) relative to the disclosed protein sequences.

The term “immune response” as used herein is defined as a cellular response to an antigen that occurs when lymphocytes identify antigenic molecules as foreign and induce the formation of antibodies and/or activate lymphocytes to remove the antigen. In some embodiments, the immune response can be against a tumor cell expressing the antigen. In some embodiments, the immune response is facilitated by a T cell expressing a chimeric antigen receptor, such as those, but not limited to, those provided herein.

The term “immunosuppressive” is used herein to refer to reducing overall immune response.

As used herein, the phrase “in vivo” in reference to a cell being transduced, transfected or transformed in vivo, refers to a cell being transduced, transfected or transformed in the subject without the cells being removed from the subject before such cells are transduced, transfected or transformed.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family that is able to infect non-dividing cells. Non-limiting examples of lentiviruses are HIV, SIV, and FIV. Vectors or viral-like particles derived from lentiviruses can be used to transduce cells and deliver genes or other molecules and have them expressed in a cell either in vitro (ex-vivo) or in vivo.

By the term “modified” as used herein, is meant a changed state or structure of a molecule or cell as provided herein. Molecules may be modified in many ways, including chemically, structurally, and functionally, such as mutations, substitutions, insertions, or deletions (e.g. internal deletions truncations). Cells may be modified through the introduction of nucleic acids or the expression of heterologous proteins.

By the term “modulating,” as used herein, is meant mediating an increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as, a human.

As used herein, the following abbreviations for the commonly occurring nucleic acid bases are used: “A” refers to adenosine, “C” refers to cytosine, “G” refers to guanosine, “T” refers to thymidine, and “U” refers to uridine.

The “Nipah virus” (NiV) is member of the family Paramyxoviridae, genus Henipavirus. Nipah virus is an enveloped virus with negative-stranded polarity and a non-segmented RNA genome consisting of helical nucleocapsids. Two strains of Nipah virus include, but are not limited to, the Malaysian (MY) and the Bangladesh (BD) strains.

The “Measles virus” (MeV) is a member of the family Paramyxoviridae, genus Morbillivirus. Measles virus is a single-stranded, negative-sense, enveloped, non-segmented RNA virus. The two envelope glycoproteins of the viral surface are the humagglutinin (H) and membrane fusion (F) proteins. The H protein mediates receptor attachment and the F protein causes fusion of the viral envelope and cellular membrane.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “oligonucleotide” typically refers to short polynucleotides. It will be understood that when a nucleotide sequence is represented by a DNA sequence (i.e., A, T, C, G), this also provides the corresponding RNA sequence (i.e., A, U, C, G) in which “U” replaces “T.”

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, the terms “nucleic acids” and “polynucleotides” as used herein are interchangeable. As used herein polynucleotides include, but are not limited to, all nucleic acid sequences which are obtained by any methods available in the art, including, without limitation, recombinant methods, i.e., the cloning of nucleic acid sequences from a recombinant library or a cell genome, using cloning technology and PCR, and the like, and by synthetic means.

As used herein, the terms “peptide,” “polypeptide,” and “protein” are used interchangeably, and refer to a compound comprised of a plurality of amino acid residues covalently linked by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “pseudotyped” or “pseudotyped viral particle”, as used herein, refers to a viral particle bearing glycoproteins derived from other viruses having envelopes or a viral vector encoding envelope glycoproteins from a virus that is different from the parental virus. The host range of the vector particles can thus be expanded or altered depending on the type of cell surface receptor used by the glycoprotein. For example, a HIV lentiviral vector can have the HIV envelope glycoprotein be replaced with the another virus's glycoprotein. For example, the envelope glycoprotein of the Nipah virus can be used. Therefore, in some embodiments, the viral particle is encoded by a lentivirus that encodes the Nipah viral envelope glycoprotein. In some embodiments, the Nipah viral envelope glycoprotein is glycoprotein F or as otherwise provided for herein. In some embodiments, the Nipah viral envelope glycoprotein is glycoprotein G. In some embodiments, the pseudotyped viral vector encodes both the Nipah viral glycoprotein F and glycoprotein G. In some embodiments, the pseudotyped viral particle expresses one or both of the Nipah viral glycoprotein F and glycoprotein G. Other embodiments of pseudotyping are also provided for herein and can be used.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” includes living organisms, including those in which an immune response can be elicited (e.g., mammals). A “subject” or “patient,” as used therein, may be a human or non-human mammal. Non-human mammals include, for example, livestock and pets, such as ovine, bovine, porcine, canine, non-human primates, feline and murine mammals. In some embodiments, the subject is human.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into a cell. A “transfected” or “transformed” or “transduced” cell is one which has been transfected, transformed or transduced with exogenous nucleic acid. The cell includes the primary subject cell and its progeny. In some embodiments, the transfection, transformation, or transduction is performed or occurs in vivo.

To “treat” a disease as the term is used herein, means to reduce the frequency or severity of at least one sign or symptom of a disease or disorder experienced by a subject.

The term “truncated” as used herein in reference to a protein sequence refers to a protein that has either, or both, portions of the N-terminus or C-terminus protein not present (e.g. deleted). If a protein comprises a truncated protein, it is meant to exclude the full-length protein and a truncated protein does not encompass the full-length protein.

As used herein, the term “variant” when used in conjunction to an amino acid sequence refers to a sequence that is at least, or about, 85%, 90%, 91%, 92%, 93%, 94%, 95%, 96%, 97%, 98%, or 99% identical to the reference sequence. In some embodiments, the variant comprises 1, 2, 3, 4, 5, 6, 7, 8, 9, or 10 substitutions. In some embodiments, the substitution is a conservative substitution.

A “vector” is a composition of matter which comprises an isolated nucleic acid encoding a protein or a peptide. Numerous vectors are known in the art including, but not limited to, linear polynucleotides, plasmids, DNA, and RNA. Examples of viral vectors include, but are not limited to, Sendai viral vectors, adenoviral vectors, adeno-associated virus vectors, retroviral vectors, lentiviral vectors, and the like.

A “carrier” or “delivery vehicle” includes viral particles, viruses, polylysine compounds, and liposomes, which facilitate transfer of nucleic acid into cells. A carrier or delivery vehicle can also be used to deliver a protein or peptide to a cell.

Ranges: throughout this disclosure, various aspects of the embodiments can be presented in a range format. It should be understood that the description in range format is merely for convenience and brevity and should not be construed as an inflexible limitation. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Unless otherwise explicitly stated to the contrary, a range that is disclosed also includes the endpoints of the range.

Without being bound to any particular theory, the embodiments provided for herein have been found to increase transduction efficiency of a virus that comprises a targeting polypeptide and a fusion polypeptide that allows a virus to infect in a cell. Previously, a virus was prepared that only had a single type of a G like glycoprotein, wherein the G protein (or could be H protein) and also had a targeting moiety linked to the G or H protein. However, the transduction efficiency of such a virus could still be improved. By adding another G or H glycoprotein that does not comprise a targeting moiety it was found that such a virus or viral like particle had increased transduction efficiency as compared to the virus with only the targeted G/H protein. Therefore, the embodiments provided herein can be used as described, including, but not limited to increase transduction efficiency and expression of a cargo of interest. These results were surprising and unexpected.

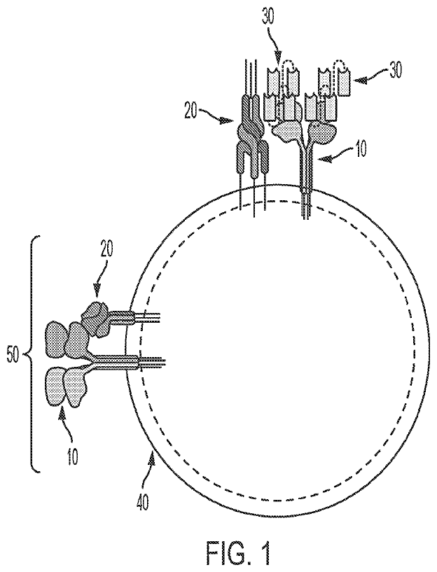

Without being bound to any particular theory, the virus can be illustrated, in part, as shown in FIG. 1. FIG. 1 illustrates the exterior of an enveloped virus (40) (the interior of the virus is not shown in this non-limiting example). A glycoprotein G or H of a virus of the Paramyxoviridae family (10) is shown either linked to a targeting moiety (30) or without a targeting moiety. The glycoprotein G or H of a virus of the Paramyxoviridae family (10) is also illustrated as interacting with a glycoprotein derived from an envelope glycoprotein F of a virus of the Paramyxoviridae family (20). Without being bound to any particular theory, it is thought that the glycoprotein G or H of a virus of the Paramyxoviridae family (10) without a targeting moiety is able to stabilize the glycoprotein derived from an envelope glycoprotein F of a virus of the Paramyxoviridae family (20) thereby facilitating an increase in transduction efficiency. (50) illustrates a non-limiting example of the glycoprotein G or H of a virus of the Paramyxoviridae family (10) without a targeting moiety interacting with (bound to) the glycoprotein derived from an envelope glycoprotein F of a virus of the Paramyxoviridae family (20). For clarity, FIG. 1 is merely for illustrative purposes only and the actual mechanism that leads to an increase in transduction may be different and the proteins may interact with one another in a different way resulting in an increase in transduction or transduction efficiency.

Accordingly, provided for herein are enveloped pseudotyped viral particles. In some embodiments, pseudotyped viral-like particles or viral vectors are provided. In some embodiments, the particles or vectors comprise a targeting polypeptide comprising a heterologous targeting moiety linked to a protein derived from an envelope glycoprotein G or H of a virus of the Paramyxoviridae family; a truncated protein derived from an envelope glycoprotein G or H of a virus of the Paramyxoviridae family, wherein the truncated protein does not comprise (e.g., free of) a targeting moiety; a glycoprotein derived from an envelope glycoprotein F of a virus of the Paramyxoviridae family, wherein the targeting polypeptide and the truncated protein comprise the same or different envelope glycoproteins. In some embodiments, the viral-like particle is a retroviral-like particle or retroviral vector.

In reference to the truncated protein that does not comprise a targeting moiety, such a protein does not have a heterologous domain whose primary function is to bind to an antigen or other molecule on another cell. Thus, although the G/H and F proteins can interact with one another, this would not be considered to be a targeting moiety because they interact with one another. In contrast, as provided for herein, the G or H protein can be made to have a targeting moiety that can facilitate the targeting of the viral particle to a cell that expresses a molecule to which the targeting moiety binds to. Therefore, a truncated protein that is free of a targeting moiety can also be referred to as a peptide that does not comprise, contain or have a heterologous targeting moiety.

The envelope glycoprotein G or H of a virus of the Paramyxoviridae family of the targeting polypeptide can be a modified envelope glycoprotein G or H of a virus of the Paramyxoviridae family. In some embodiments, the modified envelope glycoprotein G or H of a virus of the Paramyxoviridae family is a mutated envelope glycoprotein G or H (e.g. comprises truncations, deletions, insertions, or point mutations). In some embodiments, the modified envelope glycoprotein G or H of a virus of the Paramyxoviridae family comprises at least one mutation (e.g. comprises at least one truncation, deletion, insertion, point mutation, or combination thereof). In some embodiments, the modified envelope glycoprotein G or H of a virus does not bind to its native target protein.

In some embodiments, the virus of the Paramyxoviridae family is a henipavirus. In some embodiments, the virus of the Henipavirus genus is Nipah virus, Cedar virus, or Hendra virus. In some embodiments, the virus of the Henipavirus genus is Nipah virus (“NiV”).

In some embodiments, the targeting polypeptide and the truncated peptide, each independently, comprise a NiV glycoprotein G protein (“NiV-G”) comprising a N-terminal deletion. In some embodiments, the targeting polypeptide and the truncated peptide each, independently, comprise a NiV glycoprotein G protein comprising at least one mutation. Non-limiting examples are provided for herein. For example, in some embodiments, the NiV glycoprotein G protein (“NiV-G protein”) comprises a deletion of the cytoplasmic portion. In some embodiments, the NiV-G protein comprises a deletion of at least 10 contiguous amino acid residues from the cytoplasmic tail. In some embodiments, the NiV-G protein comprises a deletion of at least 15 contiguous amino acid residues from the cytoplasmic tail. In some embodiments, the NiV-G protein comprises a deletion of at least 20 contiguous amino acid residues from the cytoplasmic tail. In some embodiments, the NiV-G protein comprises a deletion that is located within two amino acid residues of the N-terminus of the NiV-G protein. In some embodiments, the NiV-G protein comprises a deletion comprising amino acid residues 3-7, 3-12, 3-17, 3-22, or 3-27 of SEQ ID NO: 1 as provided below.

(SEQ ID NO: 1)
MPTESKKVRFENTASDKGKNPSKVIKSYYGIMDIKKINEGLLDSK
ILSAFNTVIALLGSIVIIVMNIMIIQNYTRSTDNQAMIKDALQSI
QQQIKGLADKIGTEIGPKVSLIDTSSTITIPANIGLLGSKISQST
ASINENVNEKCKFTLPPLKIHECNISCPNPLPFREYKPQTEGVSN
LVGLPNNICLQKTSNQILKPKLISYTLPVVGQSGTCITDPLLAMD
EGYFAYSHLEKIGSCSRGVSKQRIIGVGEVLDRGDEVPSLEMTNV
WTPSNPNTVYHCSAVYNNEFYYVLCAVSVVGDPILNSTYWSGSLM
MTRLAVKPKNNGESYNQHQFALRNIEKGKYDKVMPYGPSGIKQGD
TLYFPAVGFLVRIEFTYNDSNCPIAECQYSKPENCRLSMGIRPNS
HYILRSGLLKYNLSDEENSKIVFIEISDQRLSIGSPSKIYDSLGQ
PVFYQASFSWDTMIKFGDVQTVNPLVVNWRDNTVISRPGQSQCPR
FNKCPEVCWEGVYNDAFLIDRINWISAGVELDSNQTAENPVFTVE
KDNEVLYRAQLASEDTNAQKTITNCFLLKNKIWCISLVEIYDTGD
NVIRPKLFAVKIPEQCT.

In some embodiments, the NiV-G protein comprises a cytoplasmic tail truncation consisting or comprising of deletion of amino acid residues 1-34 of SEQ ID NO: 1, as shown in underline above. In some embodiments, NiV-G protein comprises an amino acid sequence having at least 90%, 91%, 92%, 93%, 94%, 95%, 96%, 97%, 98%, 99%, or 100% identity to SEQ ID NO: 2 as provided below.

(SEQ ID NO: 2)
MKKINEGLLDSKILSAFNIVIALLGSIVIIVMNIMIIQNYTRSTD
NQAMIKDALQSIQQQIKGLADKIGTEIGPKVSLIDTSSTITIPAN
IGLLGSKISQSTASINENVNEKCKFTLPPLKIHECNISCPNPLPF
REYKPQTEGVSNLVGLPNNICLQKISNQILKPKLISYTLPVVGQS
GTCITDPLLAMDEGYFAYSHLEKIGSCSRGVSKQRIIGVGEVLDR
GDEVPSLFMTNVWTPSNPNTVYHCSAVYNNEFYYVLCAVSVVGDP
ILNSTYWSGSLMMTRLAVKPKNNGESYNQHQFALRNIEKGKYDKV
MPYGPSGIKQGDTLYFPAVGFLVRIEFTYNDSNCPIAECQYSKPE
NCRLSMGIRPNSHYILRSGLLKYNLSDEENSKIVFIEISDQRLSI
GSPSKIYDSLGQPVFYQASFSWDTMIKFGDVQTVNPLVVNWRDNT
VISRPGQSQCPRFNKCPEVCWEGVYNDAFLIDRINWISAGVFLDS
NQTAENPVFTVFKDNEVLYRAQLASEDINAQKTITNCFLLKNKIW
CISLVEIYDIGDNVIRPKLFAVKIPEQCT.

In some embodiments, the glycoprotein derived from an envelope glycoprotein F of a virus of the Paramyxoviridae family comprises a cytoplasmic tail deletion of the F protein. In some embodiments, the glycoprotein derived from an envelope glycoprotein F of a virus of the Paramyxoviridae family comprises a cytoplasmic tail deletion of the F protein, wherein the full length F protein comprises the sequence of SEQ ID NO: 3 as provided below.

(SEQ ID NO: 3)
MVVILDKRCYCNLLILILMISECSVGILHYEKLSKIGLVKGVTRK
YKIKSNPLTKDIVIKMIPNVSNMSQCTGSVMENYKTRLNGILTPI
KGALEIYKNNTHDLVGDVRLAGVIMAGVAIGIATAAQITAGVALY
EAMKNADNINKLKSSIESTNEAVVKLQETAEKTVYVLTALQDYIN
TNLVPTIDKISCKQTELSLDLALSKYLSDLLFVFGPNLQDPVSNS
MTIQAISQAFGGNYETLLRTLGYATEDFDDLLESDSITGQIIYVD
LSSYYIIVRVYFPILTEIQQAYIQELLPVSFNNDNSEWISIVPNF
ILVRNTLISNIEIGFCLITKRSVICNQDYATPMINNMRECLIGST
EKCPRELVVSSHVPRFALSNGVLFANCISVTCQCQTTGRAISQSG
EQTLLMIDNITCPTAVLGNVIISLGKYLGSVNYNSEGIAIGPPVF
TDKVDISSQISSMNQSLQQSKDYIKEAQRLLDTVNPSLISMLSMI
ILYVLSIASLCIGLITFISFIIVEKKRNTYSRLEDRRVRPTSSGD
LYYIGT.

In some embodiments, the glycoprotein derived from an envelope glycoprotein F of a virus of the Paramyxoviridae family is a Nipah F protein (“NiV-F protein”). In some embodiments, the NiV-F protein has a cytoplasmic tail truncation comprising the deletion of amino acid residues 526-546 of SEQ ID NO: 3. In some embodiments, the NiV-F protein comprises a cytoplasmic tail lacking amino residues 525-544 of SEQ ID NO: 3. In some embodiments, the cytoplasmic tail of the NiV-F protein is truncated after amino acid residue 524 of SEQ ID NO: 3. In some embodiments, the cytoplasmic tail of the NiV-F protein is truncated after amino acid residue 525 of SEQ ID NO: 3. In some embodiments, the cytoplasmic tail of the NiV-F protein is truncated after amino acid residue 526 of SEQ ID NO: 3. In some embodiments, the NiV-F protein comprises a substitution of glutamine for asparagine at an amino acid position that corresponds to position 99 of SEQ ID NO: 3. In some embodiments, the NiV-F protein comprises an amino acid sequence having at least 90%, 91%, 92%, 93%, 94%, 95%, 96%, 97%, 98%, 99%, or 100% identity to SEQ ID NO: 4 as provided below.

(SEQ ID NO: 4)
MVVILDKRCYCNLLILILMISECSVGILHYEKLSKIGLVKGVTRK
YKIKSNPLTKDIVIKMIPNVSNMSQCTGSVMENYKIRLNGILTPI
KGALEIYKNNTHDLVGDVRLAGVIMAGVAIGIATAAQITAGVALY
EAMKNADNINKLKSSIESTNEAVVKLQETAEKTVYVLTALQDYIN
INLVPTIDKISCKQTELSLDLALSKYLSDLLFVFGPNLQDPVSNS
MTIQAISQAFGGNYETLLRTLGYATEDFDDLLESDSITGQIIYVD
LSSYYIIVRVYFPILTEIQQAYIQELLPVSENNDNSEWISIVPNF
ILVRNTLISNIEIGFCLITKRSVICNQDYATPMINNMRECLTGST
EKCPRELVVSSHVPRFALSNGVLFANCISVTCQCQTTGRAISQSG
EQTLLMIDNITCPTAVLGNVIISLGKYLGSVNYNSEGIAIGPPVF
TDKVDISSQISSMNQSLQQSKDYIKEAQRLLDTVNPSLISMLSMI
ILYVLSIASLCIGLITFISFIIVEKKRNT.

In some embodiments, pseudotyped viral particles or viral vectors are provided that comprise a targeting polypeptide that comprises a heterologous targeting moiety linked to a NiV-G; a truncated protein that comprises a mutated NiV-G protein that does not comprise (e.g., free of) a targeting moiety; and a glycoprotein derived from a NiV-F protein, wherein the targeting polypeptide and the truncated protein comprise the same or different NiV-G protein. In some embodiments, the targeting polypeptide and the truncated protein comprise the same NiV-G protein. In some embodiments, the targeting polypeptide and the truncated protein, each independently, comprise a NiV glycoprotein G protein (“NiV-G”) comprising a N-terminal deletion. In some embodiments, the targeting polypeptide and the truncated protein, each independently, comprise a NiV glycoprotein G protein (“NiV-G protein”) comprising a deletion of the cytoplasmic portion. In some embodiments, the targeting polypeptide and the truncated protein, each independently, comprise a NiV-G protein comprising a deletion of at least 10 contiguous amino acid residues from the cytoplasmic tail. In some embodiments, the targeting polypeptide and the truncated protein, each independently, comprise a NiV-G protein comprising a deletion of at least 15 contiguous amino acid residues from the cytoplasmic tail. In some embodiments, the targeting polypeptide and the truncated protein, each independently, comprise a NiV-G protein comprising a deletion of at least 20 contiguous amino acid residues from the cytoplasmic tail. In some embodiments, the targeting polypeptide and the truncated protein, each independently, comprise a NiV-G protein comprising NiV-G protein deletion is located within two amino acid residues of the N-terminus of the NiV-G protein. In some embodiments, the targeting polypeptide and the truncated protein, each independently, comprise a NiV-G protein comprising a deletion comprising amino acid residues 3-7, 3-12, 3-17, 3-22, or 3-27 of SEQ ID NO: 1. In some embodiments, the targeting polypeptide and the truncated protein, each independently, comprise a NiV-G protein comprising a cytoplasmic tail truncation consisting of deletion of amino acid residues 1-34 of SEQ ID NO: 1. In some embodiments, the targeting polypeptide and the truncated protein, each independently, comprise a NiV-G protein comprising an amino acid sequence having at least 90%, 91%, 92%, 93%, 94%, 95%, 96%, 97%, 98%, 99%, or 100% identity to SEQ ID NO: 2.

In some embodiments, the particle that comprises the targeting polypeptide and the truncated polypeptide comprising a NiV-G protein as provided for herein, comprises a Nipah-F protein (“NiV-F protein”) or a protein derived from the same. In some embodiments, the NiV-F protein has a cytoplasmic tail truncation comprising the deletion of amino acid residues 526-546 of SEQ ID NO: 3. In some embodiments, the NiV-F protein comprises a cytoplasmic tail lacking amino residues 525-544 of SEQ ID NO: 3. In some embodiments, the cytoplasmic tail of the NiV-F protein is truncated after amino acid residue 524 of SEQ ID NO: 3. In some embodiments, the cytoplasmic tail of the NiV-F protein is truncated after amino acid residue 525 of SEQ ID NO: 3. In some embodiments, the cytoplasmic tail of the NiV-F protein is truncated after amino acid residue 526 of SEQ ID NO: 3.

In some embodiments, pseudotyped viral-like particles or viral vectors comprise envelope glycoproteins of the Paramyxoviridae family morbillivirus. Accordingly, in some embodiments, the glycoprotein derived from an envelope glycoprotein F of a virus of the Paramyxoviridae family is MeV-F. In some embodiments, the MeV-F protein comprises an amino acid sequence having at least 90%, 91%, 92%, 93%, 94%, 95%, 96%, 97%, 98%, 99%, or 100% identity to SEQ ID NO: 5. In some embodiments, the MeV-F protein comprises a truncated cytoplasmic portion. In some embodiments, the truncated cytoplasmic portion of the F protein comprises at least 1 positively charged amino acid residue and no more than 9 consecutive amino acid residues as counted from the N-terminal end of the cytoplasmic portion of the F protein. In some embodiments, the measles F protein with a truncated cytoplasmic portion comprises:

(SEQ ID NO: 5)
MGLKVNVSAIFMAVLLTLQTPTGQIHWGNLSKIGVVGIGSASYKV
MTRSSHQSLVIKLMPNITLLNNCTRVEIAEYRRLLRTVLEPIRDA
LNAMTQNIRPVQSVASSRRHKRFAGVVLAGAALGVATAAQITAGI
ALHQSMLNSQAIDNLRASLETINQAIEAIRQAGQEMILAVQGVQD
YINNELIPSMNQLSCDLIGQKLGLKLLRYYTEILSLFGPSLRDPI
SAEISIQALSYALGGDINKVLEKLGYSGGDLLGILESRGIKARIT
HVDTESYLIVLSIAYPTLSEIKGVIVHRLEGVSYNIGSQEWYTTV
PKYVATQGYLISNFDESSCTFMPEGTVCSQNALYPMSPLLQECLR
GSTKSCARTLVSGSFGNRFILSQGNLIANCASILCKCYTTGTIIN
QDPDKILTYIAADHCPVVEVNGVTIQVGSRRYPDAVYLHRIDLGP
PILLERLDVGTNLGNAIAKLEDAKELLESSDQILRSMKGLSSTCI
VYILIAVCLGGLIGIPALICCCRGR.

In some embodiments, pseudotyped viral-like particles or viral vectors as provided herein comprise a targeting polypeptide and the truncated protein, each independently, comprise a MeV-H protein. In some embodiments, the MeV-H protein comprises a truncated MeV-H protein. In some embodiments, the truncated MeV-H protein comprises HcΔ14, HcΔ15, HcΔ16, HcΔ17, HcΔ18, HcΔ19, HcΔ20, HcΔ21+A and HcΔ24+4A as described in U.S. Pat. No. 10,415,057, which is hereby incorporated by reference.

In some embodiments, HcΔ14 refers to a truncated MeV-H protein wherein 14 residues of the cytoplasmic portion have been deleted. In some embodiments, HcΔ14 refers to a truncated MeV-H wherein amino acids 2-15 of MeV-H have been deleted. In some embodiments, HcΔ15 refers to a truncated MeV-H protein wherein 15 residues of the cytoplasmic portion have been deleted. In some embodiments, HcΔ15 refers to a truncated MeV-H wherein amino acids 2-16 of MeV-H have been deleted. In some embodiments, HcΔ16 refers to a truncated MeV-H protein wherein 16 residues of the cytoplasmic portion have been deleted. In some embodiments, HcΔ16 refers to a truncated MeV-H wherein amino acids 2-17 of MeV-H have been deleted. In some embodiments, HcΔ17 refers to a truncated MeV-H protein wherein 17 residues of the cytoplasmic portion have been deleted. In some embodiments, HcΔ17 refers to a truncated MeV-H wherein amino acids 2-18 of MeV-H have been deleted. In some embodiments, HcΔ18 refers to a truncated MeV-H protein wherein 18 residues of the cytoplasmic portion have been deleted. In some embodiments, HcΔ18 refers to a truncated MeV-H wherein amino acids 2-19 of MeV-H have been deleted. In some embodiments, HcΔ19 refers to a truncated MeV-H protein wherein 19 residues of the cytoplasmic portion have been deleted. In some embodiments, HcΔ19 refers to a truncated MeV-H wherein amino acids 2-20 of MeV-H have been deleted. In some embodiments, HcΔ20 refers to a truncated MeV-H protein wherein 20 residues of the cytoplasmic portion have been deleted. In some embodiments, HcΔ20 refers to a truncated MeV-H wherein amino acids 2-21 of MeV-H have been deleted.

In some embodiments, HcΔ21+A refers to a truncated MeV-H protein wherein 21 residues of the cytoplasmic portion have been deleted and an alanine has been inserted at the N-terminal portion of the remaining cytoplasmic portion. In some embodiments, HcΔ21+A refers to a truncated MeV-H protein wherein amine acids 2-22 of MeV-H have been deleted and an alanine has been inserted at the N-terminal portion of the remaining cytoplasmic portion. Accordingly, in some embodiments, HcΔ21+A may have the formula M-A-(MeV-H AA23), wherein M is methionine, A is the inserted alanine, and MeV-H AA23 is the 23^{rd} amino acid of the MeV-H full length protein. In some embodiments, HcΔ24+4A refers to a truncated MeV-H protein wherein 24 residues of the cytoplasmic portion have been deleted and four alanine residues have been inserted at the N-terminal portion of the remaining cytoplasmic portion. In some embodiments, HcΔ24+4A refers to a truncated MeV-H protein wherein amine acids 2-25 of MeV-H have been deleted and four alanine residues have been inserted at the N-terminal portion of the remaining cytoplasmic portion. Accordingly, in some embodiments, HcΔ24+A may have the formula M-AAAA-(MeV-H AA26), wherein M is methionine, AAAA (SEQ ID NO: 10) are the four inserted alanine residues, and MeV-H AA26 is the 26′ amino acid of the MeV-H full length protein.

In some embodiments, the truncated MeV-H protein comprises:

(SEQ ID NO: 6)
MGSRIVINREHLMIDRPYVLLAVLFVMFLSLIGLLAIAGIRLHRA
AIYTAEIHKSLSTNLDVINSIEHQVKDVLTPLFKIIGDEVGLRTP
QRFTDLVKFISDKIKFLNPDREYDERDLTWCINPPERIKLDYDQY
CADVAAEELMNALVNSTLLETRTTNQFLAVSKGNCSGPTTIRGQF
SNMSLSLLDLYLGRGYNVSSIVTMTSQGMYGGTYLVEKPNLSSKR
SELSQLSMYRVFEVGVIRNPGLGAPVFHMTNYLEQPVSNDLSNCM
VALGELKLAALCHGEDSITIPYQGSGKGVSFQLVKLGVWKSPTDM
QSWVPLSTDDPVIDRLYLSSHRGVIADNQAKWAVPTTRIDDKLRM
ETCFQQACKGKIQALCENPEWAPLKDNRIPSYGVLSVDLSLIVEL
KIKIASGFGPLITHGSGMDLYKSNHNNVYWLTIPPMKNLALGVIN
TLEWIPRFKVSPALFNVPIKEAGEDCHAPTYLPAEVDGDVKLSSN
LVILPGQDLQYVLATYDTSAVEHAVVYYVYSPSRLSSYFYPFRLP
IKGVPIELQVECFTWDQKLWCRHFCVLADSESGGHITHSGMVGMG
VSCTVTREDGTNRRGG.

In some embodiments, the targeting moiety can be used to direct the viral particle to a specific cell target. The targeting moiety can be any cell surface protein that imparts selectivity to the pseudotyped viral particle and allows the viral particle to infect the specific target cell. In some non-limiting embodiments, the targeting moiety is an antibody, a scFv antibody, an antigen binding domain, an ankyrin repeat (e.g. DARPIN), a VHH domain antibody, a nanobody, single domain antibody, a FN3 domain, or any combination thereof. The targeting moiety can bind to a target on the target cell.

In some embodiments, the target cell is an immune cell, such as, but not limited to, T cell, B cell; NK cell, dendritic cell, neutrophils, macrophages, a cancer cell; or, for example, CD3+ T cell; CD4+ T cell; CD7+ T cell, CD8+ T cell; CD19+ B cell; CD19+ cancer cell; CD20+ B cell; CD30+ lung epithelial cell; CD34+ haematopoietic stem cell; CD105+ endothelial cell; CD105+ haematopoietic stem cell; CD117+ haematopoietic stem cell; CD133+ cancer cell; EpCAM+ cancer cell; GluA2+ neuron; GluA4+ neuron; Haematopoietic stem cell; Hepatocyte; Her2/Neu+ cancer cell; NKG2D+ natural killer cell; SLC1A3+ astrocyte; SLC7A10+ adipocyte. In some embodiments, the target cell is a CD7+ T cell and/or a CD8+ T cell. In some embodiments, the cell is an immune cell. In some embodiments, the target cell is a T cell. In some embodiments, the target cell is a NK cell. In some embodiments, the target cell is a B cell. In some embodiments, the target cell is a dendritic cell. In some embodiments, the target cell is a macrophage.

In some embodiments, the targeting moiety is selected from the group consisting of Stem Cell Factor protein (SCF, KIT-ligand, KL, or steel factor) or a moiety that binds to cKit (CD117), CD4, CD8, CD3, CD5, CD6, CD7, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, and CXCR3. In some embodiments, the targeting moiety binds to CD7 or CD8.

In some embodiments, the pseudotyped viral particle further comprises a heterologous nucleic acid molecule encoding a cargo of interest. The nucleic acid molecule may be useful for modulating the expression of a target gene. Therefore, in some embodiments, the nucleic acid may comprise an siRNA or an shRNA. The nucleic acid may also encode for a cargo of interest. Therefore, in some embodiments, the cargo of interest may comprise a polypeptide or portion thereof, a protein or portion thereof, a chimeric antigen receptor or portion thereof, or a tumor antigen or a portion thereof. In some embodiments, the cargo of interest is an antibody that is produced by the virus, which can then be secreted by the cell that is infected with the virus. The term “protein” can refer to any polypeptide that carries a native function in a cellular environment. Therefore, in some embodiments, the protein encoded by the nucleic acid cargo of interest may comprise an enzyme, a nuclear receptor, a transporter, a ribosomal protein, a membrane bound protein, a cytoplasmic protein, a G-protein coupled receptor, a voltage gated ion channel, a secretory protein, a mitochondria protein, a cytokine, a chimeric antigen receptor, a tumor antigen, or a portion or chimeric species thereof.

In some embodiments, non-limiting examples of targeting polypeptides of the present disclosure are provided herein. In some embodiments, the non-limiting examples of targeting polypeptides comprising Nipah G linked via a linker to a targeting moiety, such as a targeting moiety that binds CD7, are provided in SEQ ID NO: 7. In some embodiments, the exemplary polypeptide molecule comprises a restriction sequence, such as a DI sequence as shown in SEQ ID NO: 7 (bold, italics, and underline). In some embodiments, the restriction sequence is absent. In some embodiments, the restriction sequence comprises a sequence that is not DI. In some embodiments, the restriction sequence comprises a sequence that is random. In some embodiments, the linker is a peptide linker, such as, but not limited to, those provided herein. In some embodiments, the restriction sequence comprises a random sequence.

(SEQ ID NO: 7)
MKKINEGLLDSKILSAFNIVIALLGSIVIIVMNIMIIQNYTRSTD
NQAMIKDALQSIQQQIKGLADKIGTEIGPKVSLIDTSSTITIPAN
IGLLGSKISQSTASINENVNEKCKFTLPPLKIHECNISCPNPLPF
REYKPQTEGVSNLVGLPNNICLQKTSNQILKPKLISYTLPVVGQS
GTCITDPLLAMDEGYFAYSHLEKIGSCSRGVSKQRIIGVGEVLDR
GDEVPSLFMTNVWTPSNPNTVYHCSAVYNNEFYYVLCAVSVVGDP
ILNSTYWSGSLMMTRLAVKPKNNGESYNQHQFALRNIEKGKYDKV
MPYGPSGIKQGDTLYFPAVGFLVRIEFTYNDSNCPIAECQYSKPE
NCRLSMGIRPNSHYILRSGLLKYNLSDEENSKIVFIEISDQRLSI
GSPSKIYDSLGQPVFYQASFSWDTMIKFGDVQTVNPLVVNWRDNT
VISRPGQSQCPRFNKCPVVCAEGVYNDAFLIDRINWISAGVELDS
NATAANPVFTVFKDNEVLYRAQLASEDINAQKTITNCFLLKNKIW
CISLVEIYDTGDNVIRPKLFAVKIPEQCTDIGGGGSGGGGSGGGG
SGGGGSDIELTQSPATLSVTPGDSVSLSCRASQSISNNLHWYQQK
SHESPRLLIKSASQSISGIPSRFSGSGSGTDFTLSINSVETEDFG
MYFCQQSNSWPYTFGGGTKLEIKRGGGGSGGGGSGGGGSGGGGSQ
VQLQESGAELVKPGASVKLSCKASGYTFTSYWMHWVKQRPGQGLE
WIGKINPSNGRINYNEKFKSKATLTVDKSSSTAYMQLSSLTSEDS
AVYYCARGGVYYDLYYYALDYWGQGTTVTVSS

In some embodiments, the targeting polypeptide comprises a polypeptide having a general formula of: [PG]-[RR]-Linker-[TM], wherein
PG is an envelope glycoprotein G or H sequence of a virus of the Paramyxoviridae family, such as, but not limited to, as provided herein;
RR amino acid sequence is an optional restriction site sequence, such as, but not limited to, as provided herein;
Linker is a peptide linker, such as, but not limited to, those provided herein; and
TM is a targeting moiety, such as, but not limited to, as provided herein.

In some embodiments, the PG comprises a Nipah G protein. In some embodiments, the Nipah G amino acid sequence comprises the full-length amino acid sequence or a truncated amino acid sequence. In some embodiments, the Nipah G amino acid sequence comprises SEQ ID NO: 1 or SEQ ID NO: 2. In some embodiments, the PG amino acid sequence comprises a Measles H protein. In some embodiments, the Measles H amino acid sequence comprises a full-length Measles H amino acid sequence, or a truncated Measles H amino acid sequence. In some embodiments, the truncated Measles H amino acid sequence comprises SEQ ID NO: 6. In some embodiments, the truncated Measles H amino acid sequence comprises HcΔ14, HcΔ15, HcΔ16, HcΔ17, HcΔ18, HcΔ19, HcΔ20, HcΔ21+A and HcΔ24+4A as provided for herein and as described in U.S. Pat. No. 10,415,057, which is hereby incorporated by reference.

In some embodiments, the RR amino acid sequence has the amino acid sequence of DI. In some embodiments, the RR amino acid sequence is absent. In some embodiments, the RR amino acid sequence is not DI. In some embodiments, the RR amino acid sequence is random. In some embodiments, the RR amino acid sequence is absent, i.e., it is optional.

In some embodiments, the Linker is a peptide linker. Examples of peptide linkers that can be used to link various peptides provided for herein include, but are not limited to: (GGGGS)n (SEQ ID NO: 8); or (GGGGA)n (SEQ ID NO: 9), wherein each n is, independently, 1-5. In some embodiments, the linker is absent.

In some embodiments, the TM is a targeting moiety. Examples of targeting moieties include, but are not limited to, polypeptides that bind to another molecule. Examples of such polypeptides are provided for herein. In some embodiments, the targeting moiety can be Stem Cell Factor protein (SCF, KIT-ligand, KL, or steel factor) or a moiety that binds to cKit (CD117), CD4, CD8, CD3, CD5, CD6, CD7, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, and CXCR3. In some embodiments, the targeting moiety binds to CD7 or CD8. In some embodiments, the targeting moiety binds to a glycosylated CD43 epitope expressed on acute leukemia or lymphoma but not on hematopoietic progenitors; A glycosylated CD43 epitope expressed on non-hematopoietic cancers; a kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsgl); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-la); Carbonic anhydrase IX (CA1X); Carcinoembryonic antigen (CEA); CCCTC-Binding Factor (Zinc Finger Protein)-Like (BORIS or Brother of the Regulator of Imprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECL1); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLVl-Tax; Human papilloma virus E6 (HPV E6); Human papilloma virus E7 (HPV E7); Human Telomerase reverse transcriptase (hTERT); IgE; IL13Ra2; ILl 1Ra; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-1 (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAP1; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p5³); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); X Antigen Family Member 1A (XAGE1).

In some embodiments, targeting moieties include, but are not limited to, targeting moieties that bind to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3.

In some embodiments, the pseudotyped viral-like particle or viral vector comprises a polypeptide comprising the general formula of:

[PG]-[RR]-Linker-[TM];

[EG]; and

[GF]; wherein
PG is an envelope glycoprotein G or H sequence of a virus of the Paramyxoviridae family, such as, but not limited to, as provided herein;
RR is an optional restriction site sequence, such as, but not limited to, as provided herein;
Linker is a peptide linker, such as, but not limited to, those provided herein;
TM is a targeting moiety, such as, but not limited to, as provided herein.
EG is an envelope glycoprotein G or H of a virus of the Paramyxoviridae family, such as, but not limited to, as provided herein; and
GF is an envelope glycoprotein F sequence of a virus of the Paramyxoviridae family, such as, but not limited, as provided herein.

In some embodiments, the PG amino acid sequence comprises a Nipah G protein. In some embodiments, the Nipah G amino acid sequence is the full-length amino acid sequence or the truncated amino acid sequence. In some embodiments, the PG comprises a NiV glycoprotein G protein (“NiV-G”) comprising a N-terminal deletion. In some embodiments, the PG comprises a NiV glycoprotein G protein (“NiV-G protein”) comprising a deletion of the cytoplasmic portion. In some embodiments, the NiV-G protein comprises a deletion of at least 10 contiguous amino acid residues from the cytoplasmic tail. In some embodiments, the NiV-G protein comprises a deletion of at least 15 contiguous amino acid residues from the cytoplasmic tail. In some embodiments, the NiV-G protein comprises a deletion of at least 20 contiguous amino acid residues from the cytoplasmic tail. In some embodiments, the NiV-G protein comprises a deletion comprising amino acid residues 3-7, 3-12, 3-17, 3-22, or 3-27 of SEQ ID NO: 1. In some embodiments, the NiV-G protein comprises a cytoplasmic tail truncation consisting of deletion of amino acid residues 1-34 of SEQ ID NO: 1. In some embodiments, the Nipah G amino acid sequence comprises SEQ ID NO: 1 or SEQ ID NO: 2.

In some embodiments, the PG amino acid sequence comprises a Measles H protein. In some embodiments, the Measles H amino acid sequence comprises a full-length Measles H amino acid sequence, or a truncated Measles H amino acid sequence. In some embodiments, the truncated Measles H amino acid sequence comprises or is SEQ ID NO: 6. In some embodiments, the truncated Measles H amino acid sequence comprises HcΔ14, HcΔ15, HcΔ16, HcΔ17, HcΔ18, HcΔ19, HcΔ20, HcΔ21+A and HcΔ24+4A as provided for herein and as described in U.S. Pat. No. 10,415,057, which is hereby incorporated by reference.

In some embodiments, the RR amino acid sequence has the amino acid sequence of DI. In some embodiments, the RR amino acid sequence is absent. In some embodiments, the RR amino acid sequence is not DI. In some embodiments, the RR amino acid sequence is random.

In some embodiments, the Linker amino acid sequence is a peptide amino acid sequence. Examples of peptide linkers that can be used to link various peptides provided for herein include, but are not limited to: (GGGGS)n (SEQ ID NO: 8); or (GGGGA)n (SEQ ID NO: 9), wherein each n is independently 1-5. In some embodiments, the linker is absent.

In some embodiments, the TM is targeting moiety. In some embodiments, non-limiting examples of targeting moieties include targeting moieties that bind to A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsgl); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-la); Carbonic anhydrase IX (CA1X); Carcinoembryonic antigen (CEA); CCCTC-Binding Factor (Zinc Finger Protein)-Like (BORIS or Brother of the Regulator of lmprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECL1); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLVI-Tax; Human papilloma virus E6 (HPV E6); Human papilloma virus E7 (HPV E7); Human Telomerase reverse transcriptase (hTERT); IgE; IL13Ra2; ILl 1Ra; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-1 (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAP1; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p5³); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); X Antigen Family Member 1A (XAGE1).

In some embodiments, non-limiting examples of targeting moieties include targeting moieties that bind to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3. In some embodiments, the targeting moiety binds to CD7 or CD8.

In some embodiments, the EG amino acid sequence comprises a Nipah G. In some embodiments, the Nipah G amino acid sequence comprises the full-length amino acid sequence or the truncated amino acid sequence. In some embodiments, the Nipah G amino acid sequence comprises SEQ ID NO: 1 or SEQ ID NO: 2. In some embodiments, the EG sequence comprises an amino acid sequence having at least 90%, 91%, 92%, 93%, 94%, 95%, 96%, 97%, 98%, 99%, or 100% identity to SEQ ID NO: 2.

In some embodiments, the EG amino acid sequence comprises a Measles H protein. In some embodiments, the Measles H amino acid sequence comprises or is the full-length Measles H amino acid sequence, or comprises or is a truncated Measles H amino protein. In some embodiments, the truncated Measles H amino acid sequence comprises or is as provided in SEQ ID NO: 6. In some embodiments, the truncated Measles H amino acid sequence comprises HcΔ14, HcΔ15, HcΔ16, HcΔ17, HcΔ18, HcΔ19, HcΔ20, HcΔ21+A and HcΔ24+4A as provided for herein and as described in U.S. Pat. No. 10,415,057, which is hereby incorporated by reference.

In some embodiments, the GF sequence comprises a sequence derived from an envelope glycoprotein F of a virus of the Paramyxoviridae family comprises a cytoplasmic tail deletion of the F protein. In some embodiments, the GF sequence comprises a sequence derived from an envelope glycoprotein F of a virus of the Paramyxoviridae family comprises is a Nipah F protein (“NiV-F protein”). In some embodiments, the Nipah F amino acid sequence is the full-length amino acid sequence or the truncated amino acid sequence. In some embodiments, the Nipah F amino acid sequence is that of SEQ ID NO: 3 or SEQ ID NO: 4. In some embodiments, the GF sequence comprises a sequence derived from an envelope glycoprotein F of a virus of the Paramyxoviridae family comprises a cytoplasmic tail deletion of the F protein, wherein the full length F protein comprises the sequence of SEQ ID NO: 3. In some embodiments, the NiV-F protein has a cytoplasmic tail truncation comprising the deletion of amino acid residues 526-546 of SEQ ID NO: 3. In some embodiments, the NiV-F protein comprises a cytoplasmic tail lacking amino residues 525-544 of SEQ ID NO: 3. In some embodiments, the cytoplasmic tail of the NiV-F protein is truncated after amino acid residue 524 of SEQ ID NO: 3. In some embodiments, the cytoplasmic tail of the NiV-F protein is truncated after amino acid residue 525 of SEQ ID NO: 3. In some embodiments, the cytoplasmic tail of the NiV-F protein is truncated after amino acid residue 526 of SEQ ID NO: 3. In some embodiments, the NiV-F protein comprises a substitution of glutamine for asparagine at an amino acid position that corresponds to position 99 of SEQ ID NO: 3. In some embodiments, the NiV-F protein comprises an amino acid sequence having at least 90%, 91%, 92%, 93%, 94%, 95%, 96%, 97%, 98%, 99%, or 100% identity to SEQ ID NO: 4.

In some embodiments, the GF sequence is a sequence derived from an envelope glycoprotein F of a virus of the Paramyxoviridae family is MeV-F. In some embodiments, the GF amino acid sequence comprises a MeV-F protein. In some embodiments, the Measles F amino acid sequence is as provided in SEQ ID NO: 5. In some embodiments, the MeV-F protein comprises an amino acid sequence having at least 90%, 91%, 92%, 93%, 94%, 95%, 96%, 97%, 98%, 99%, or 100% identity to SEQ ID NO: 5. In some embodiments, the MeV-F protein comprises a truncated cytoplasmic portion. In some embodiments, the truncated cytoplasmic portion of the F protein comprises at least 1 positively charged amino acid residue and no more than 9 consecutive amino acid residues as counted from the N-terminal end of the cytoplasmic portion of the F protein. In some embodiments, the truncated cytoplasmic portion of the measles F protein comprises SEQ ID NO: 5.

In some embodiments, the pseudotyped viral particle is a recombinant lentivirus. In some embodiments, the recombinant pseudotyped viral particle is replication competent. In some embodiments, the recombinant pseudotyped viral particle is replication incompetent.

In some embodiments, a pharmaceutical composition is provided comprising the envelope pseudotyped viral particles or vectors as provided for herein.

In some embodiments, method of delivering a cargo of interest to a cell are provided. In some embodiments, the methods comprise contacting the cell with the pseudotyped viral-like particles or viral vectors as provided for herein, or a pharmaceutical composition comprising the same.

In some embodiments, methods of delivering a cargo of interest to a cell in a subject are provided. In some embodiments, the methods comprise administering to the subject the pseudotyped viral-like particles or viral vectors as provided for herein, or a pharmaceutical composition comprising the same. In some embodiments, the cargo is a chimeric antigen receptor or as otherwise provided for herein.

In some embodiments, methods for of delivering a chimeric antigen receptor to a T-cell in a subject are provided. In some embodiments, the methods comprising administering to the subject the pseudotyped viral-like particles or viral vectors as provided for herein, or a pharmaceutical composition comprising the same, wherein the pseudotyped viral-like particle or viral vector comprises a heterologous nucleic acid molecule encoding the chimeric antigen receptor.

Also provided herein are nucleic acid molecules encoding a targeting polypeptide comprising a heterologous targeting moiety linked to a protein derived from an envelope glycoprotein G or H of a virus of the Paramyxoviridae family; a truncated protein derived from an envelope glycoprotein G or H of a virus of the Paramyxoviridae family, wherein the truncated protein does not comprise (e.g., free of) a targeting moiety; a glycoprotein derived from an envelope glycoprotein F of a virus of the Paramyxoviridae family, wherein the targeting polypeptide and the truncated protein comprise the same or different envelope glycoproteins.

Methods of making the viral like particles or vectors are also provided. In some embodiments, the methods comprise transfecting or transducing a packaging cell line with the nucleic acid molecules provided for herein under conditions sufficient to produce the pseudotyped viral-like particles or viral vectors. In some embodiments, the methods comprise transfecting or transducing a packaging cell line with the plurality of nucleic acid molecules provided for herein under conditions sufficient to produce the pseudotyped viral-like particles or viral vectors. In some embodiments, methods further comprise isolating the pseudotyped viral-like particle or viral vector.

Also provided for herein are methods of treating cancer in a subject. In some embodiments, the methods comprise administering to the subject the pseudotyped viral-like particles or viral vectors as provided for herein, or a pharmaceutical composition comprising the same, wherein the pseudotyped viral-like particle or viral vector comprises a heterologous nucleic acid molecule encoding the chimeric antigen receptor.

Also provided herein are methods of treating a disease in a subject in need thereof.

In some embodiments, the methods provided include, but are not limited to, methods of treating a disease in a subject in need thereof, comprising administering to the subject the viral particle(s) provided herein to treat the disease.

In certain embodiments, the disease is a cancer. In addition, the compositions provided for herein can be used in methods for the treatment of any condition related to a cancer, such as a cell-mediated immune response against a tumor cell(s), where it is desirable to treat or alleviate the disease. The types of cancers to be treated include, but are not limited to, carcinoma, blastoma, sarcoma, certain leukemia or lymphoid malignancies, benign and malignant tumors, malignancies e.g., sarcomas, carcinomas, and melanomas. Other exemplary cancers include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included. In one embodiment, the cancer is a hematological tumor. In one embodiment, the cancer is a carcinoma. In one embodiment, the cancer is a sarcoma. In one embodiment, the cancer is a leukemia. In one embodiment the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by the methods disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

In certain exemplary embodiments, the compositions provided herein can be used in methods to treat a myeloma, or a condition related to myeloma. Examples of myeloma or conditions related thereto include, without limitation, light chain myeloma, non-secretory myeloma, monoclonal gamopathy of undetermined significance (MGUS), plasmacytoma (e.g., solitary, multiple solitary, extramedullary plasmacytoma), amyloidosis, and multiple myeloma. In some embodiments, methods of treating multiple myeloma are provided. In some embodiments, the multiple myeloma is refractory myeloma. In some embodiments, the multiple myeloma is relapsed myeloma.

In certain exemplary embodiments, the in vivo modified immune cells produced using the compositions provided herein are used to treat a melanoma, or a condition related to melanoma. Examples of melanoma or conditions related thereto include, without limitation, superficial spreading melanoma, nodular melanoma, lentigo maligna melanoma, acral lentiginous melanoma, amelanotic melanoma, or melanoma of the skin (e.g., cutaneous, eye, vulva, vagina, rectum melanoma). In some embodiments, the melanoma is cutaneous melanoma. In some embodiments, the melanoma is refractory melanoma. In some embodiments, the melanoma is relapsed melanoma.

In some embodiments, the compositions provided herein are used to treat a sarcoma, or a condition related to sarcoma. Examples of sarcoma or conditions related thereto include, without limitation, angiosarcoma, chondrosarcoma, chordoma, endotheliosarcoma, Ewing's sarcoma, fibrosarcoma, gastrointestinal stromal tumor, leiomyosarcoma, liposarcoma, lymphangiosarcoma, lymphangioendotheliosarcoma, mesothelioma, malignant peripheral nerve sheath tumor, myxosarcoma, osteogenic sarcoma, osteosarcoma, pleomorphic sarcoma, rhabdomyosarcoma, synovioma, synovial sarcoma, and other soft tissue sarcomas. In some embodiments, the sarcoma is synovial sarcoma. In some embodiments, the sarcoma is liposarcoma such as myxoid/round cell liposarcoma, differentiated/dedifferentiated liposarcoma, or pleomorphic liposarcoma. In some embodiments, the sarcoma is myxoid/round cell liposarcoma. In some embodiments, the sarcoma is refractory sarcoma. In some embodiments, the sarcoma is relapsed sarcoma.

In certain embodiments, the compositions are used in methods for treating sickle cell disease. In some embodiments, the viral particles are administered to a subject suffering from sickle cell disease, wherein the particles encode the hemoglobin beta chain. When expressed in the cell, the hemoglobin beta chain is expressed and alleviates the symptoms of sickle cell disease to treat the disease.

In some embodiments, the subject has been treated with a therapeutic agent targeting the disease or condition, e.g. the tumor, prior to administration of the composition. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the composition is administered prophylactically, e.g., to reduce the likelihood of or prevent relapse. In some aspects, the subject has not received prior treatment with another therapeutic agent.

The administration of the compositions may be carried out in any convenient manner known to those of skill in the art. For example, the compositions may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the compositions is injected directly into a site of a local disease site in the subject, a lymph node, an organ, a tumor, and the like.

For the prevention or treatment of disease, the appropriate dosage may depend on the type of disease to be treated, the severity and course of the disease, whether the composition is administered for preventive or therapeutic purposes, previous therapy, the subject's clinical history and response to the treatment, and the discretion of the attending physician. The composition is, in some embodiments, suitably administered to the subject at one time or over a series of treatments.

In some embodiments, the composition is administered as part of a combination treatment, such as simultaneously with or sequentially with, in any order, another therapeutic intervention, such as an antibody or produced cell or receptor or agent, such as a cytotoxic or therapeutic agent. The composition(s), in some embodiments, is co-administered with one or more additional therapeutic agents or in connection with another therapeutic intervention, either simultaneously or sequentially in any order. In some contexts, the composition is co-administered with another therapy sufficiently close in time such that the composition enhances the effect of one or more additional therapeutic agents, or vice versa. In some embodiments, the composition is administered prior to the one or more additional therapeutic agents. In some embodiments, the composition is administered after the one or more additional therapeutic agents. In some embodiments, the one or more additional agents includes a cytokine, such as IL-2, for example, to enhance persistence. In some embodiments, the methods comprise administration of a chemotherapeutic agent. In some embodiments, the methods do not comprise the administration of a chemotherapeutic agent.

In certain embodiments, the compositions may be administered to a subject in combination with an immune checkpoint antibody (e.g., an anti-PD1, anti-CTLA-4, or anti-PDL1 antibody). For example, viral vectors may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the compositions may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In certain embodiments, the composition may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the viral vector. In certain embodiments, combination treatment comprising an immune checkpoint modulator may increase the therapeutic efficacy of a therapy comprising a composition as provided herein. The other therapeutic can be administered simultaneously, before, or after the compositions provided herein are administered to the subject.

In certain embodiments, the subject is provided a secondary treatment. Secondary treatments include but are not limited to chemotherapy, radiation, surgery, and medications. In some embodiments, the subject is not provided a secondary treatment.

In some embodiments, the methods are performed without a lymphodepletion step, such as the administration of cyclophosphamide and/or fludarabine.

In some embodiments, the subject can be administered a conditioning therapy after the administration of the compositions to kill certain immune cells that are not transduced with the CAR encoded by the compositions. This can be done by including a selection marker that is encoded by the nucleic acid cargo of interest. In some embodiments, the conditioning therapy comprises administering an effective amount of cyclophosphamide to the subject. In some embodiments, the conditioning therapy comprises administering an effective amount of fludarabine to the subject. In some embodiments, the conditioning therapy comprises administering an effective amount of a combination of cyclophosphamide and fludarabine to the subject.

In some embodiments, a specific dosage regimen of the present disclosure includes a lymphodepletion step after the administration of the composition. In an exemplary embodiment, the lymphodepletion step includes administration of cyclophosphamide and/or fludarabine.

In some embodiments, the lymphodepletion step includes administration of cyclophosphamide at a dose of between about 200 mg/m2/day and about 2000 mg/m2/day (e.g., 200 mg/m2/day, 300 mg/m2/day, or 500 mg/m2/day). In an exemplary embodiment, the dose of cyclophosphamide is about 300 mg/m2/day. In some embodiments, the lymphodepletion step includes administration of fludarabine at a dose of between about 20 mg/m2/day and about 900 mg/m2/day (e.g., 20 mg/m2/day, 25 mg/m2/day, 30 mg/m2/day, or 60 mg/m2/day). In an exemplary embodiment, the dose of fludarabine is about 30 mg/m2/day.

In some embodiment, the lymphodepletion step includes administration of cyclophosphamide at a dose of between about 200 mg/m2/day and about 2000 mg/m2/day (e.g., 200 mg/m2/day, 300 mg/m2/day, or 500 mg/m2/day), and fludarabine at a dose of between about 20 mg/m2/day and about 900 mg/m2/day (e.g., 20 mg/m2/day, 25 mg/m2/day, 30 mg/m2/day, or 60 mg/m2/day). In an exemplary embodiment, the lymphodepletion step includes administration of cyclophosphamide at a dose of about 300 mg/m2/day, and fludarabine at a dose of about 30 mg/m2/day.

In an exemplary embodiment, the dosing of cyclophosphamide is 300 mg/m2/day over three days, and the dosing of fludarabine is 30 mg/m2/day over three days.

It is known in the art that one of the adverse effects of the use of CAR T cells can be the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, in some embodiments, the methods comprise, following the diagnosis of CRS, appropriate CRS management strategies to mitigate the physiological symptoms of uncontrolled inflammation without dampening the antitumor efficacy of the in vivo generated cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

In some embodiments, methods of treating cancer in a subject in need thereof are provided, the methods comprising administering to the subject any of the compositions, such as the viral particle(s), provided herein.

The compositions disclosed herein can comprise a pharmaceutical composition, and for example include a pharmaceutically acceptable carrier, and/or a pharmaceutical formulation.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the composition, preferably those with activities complementary to the composition, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the composition in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the composition, by multiple bolus administrations of the composition, or by continuous infusion administration of the composition. In some embodiments, the pharmaceutical composition does not include a chemotherapeutic.

Formulations include those for oral, intravenous, intraperitoneal, subcutaneous, pulmonary, transdermal, intramuscular, intranasal, buccal, sublingual, or suppository administration. In some embodiments, the composition is administered parenterally. The term “parenteral,” as used herein, includes intravenous, intramuscular, subcutaneous, rectal, vaginal, and intraperitoneal administration. In some embodiments, the composition is administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the composition in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

### ENUMERATED EMBODIMENTS

In some embodiments, the following embodiments are provided:
1. An pseudotyped viral-like particle or viral vector comprising:
   a targeting polypeptide comprising a heterologous targeting moiety linked to a protein derived from an envelope glycoprotein G or H of a virus of the Paramyxoviridae family;
   a truncated protein derived from an envelope glycoprotein G or H of a virus of the Paramyxoviridae family, wherein the truncated protein does not comprise a targeting moiety;
   a glycoprotein derived from an envelope glycoprotein F of a virus of the Paramyxoviridae family,
   wherein the targeting polypeptide and the truncated protein comprise the same or different envelope glycoproteins.
2. The pseudotyped viral-like particle or viral vector of embodiment 1, wherein the viral-like particle is a retroviral-like particle or retroviral vector.
3. The pseudotyped viral-like particle or viral vector of embodiment 2, wherein the retroviral-like particle is a lentivirus based viral particle or viral vector.
4. The pseudotyped viral-like particle or viral vector of any one of embodiments 1-3, wherein the envelope glycoprotein G or H of a virus of the Paramyxoviridae family of the targeting polypeptide is a modified envelope glycoprotein G or H of a virus of the Paramyxoviridae family.
5. The pseudotyped viral-like particle or viral vector of embodiment 4, wherein the modified envelope glycoprotein G or H of a virus of the Paramyxoviridae family comprises at least one mutation (e.g. comprises at least one truncation, deletion, insertion, point mutation, etc.).
6. The pseudotyped viral-like particle or viral vector of embodiment 4, wherein the modified envelope glycoprotein G or H of a virus does not bind to its native target protein.
7. The pseudotyped viral-like particle or viral vector of any one of embodiments 1-6, wherein the virus of the Paramyxoviridae family is a henipavirus.
8. The pseudotyped viral-like particle or viral vector of embodiment 7, wherein the virus of the Henipavirus genus is Nipah virus, Cedar virus, or Hendra virus.
9. The pseudotyped viral-like particle or viral vector of embodiment 7, wherein the virus of the Henipavirus genus is Nipah virus (“NiV”).
10. The pseudotyped viral particle or viral vector of embodiment 9, wherein the targeting polypeptide comprises a NiV glycoprotein G protein (“NiV-G”) comprising a N-terminal deletion.
11. The pseudotyped viral particle or viral vector of embodiment 9, wherein the targeting polypeptide comprises a NiV glycoprotein G protein (“NiV-G protein”) comprising a deletion of the cytoplasmic portion.
12. The pseudotyped viral particle or viral vector of embodiment 9, wherein the NiV-G protein comprises a deletion of at least 10 contiguous amino acid residues from the cytoplasmic tail.
13. The pseudotyped viral particle or viral vector of embodiment 9, wherein the NiV-G protein comprises a deletion of at least 15 contiguous amino acid residues from the cytoplasmic tail.
14. The pseudotyped viral particle or viral vector of embodiment 9, wherein the NiV-G protein comprises a deletion of at least 20 contiguous amino acid residues from the cytoplasmic tail.
15. The pseudotyped viral particle or viral vector of embodiment 9, wherein the NiV-G protein comprises a deletion located within two amino acid residues of the N-terminus of the NiV-G protein.
16. The pseudotyped viral particle or viral vector of embodiment 9, wherein the NiV-G protein comprises a deletion comprising amino acid residues 3-7, 3-12, 3-17, 3-22, or 3-27 of SEQ ID NO: 1.
17. The pseudotyped viral particle or viral vector of embodiment 9, wherein the NiV-G protein comprises a cytoplasmic tail truncation consisting of deletion of amino acid residues 1-34 of SEQ ID NO: 1.
18. The pseudotyped viral particle or viral vector of embodiment 8, wherein the second NiV-G protein comprises an amino acid sequence having at least 90%, 91%, 92%, 93%, 94%, 95%, 96%, 97%, 98%, 99%, or 100% identity to SEQ ID NO: 2.
19. The pseudotyped viral particle or viral vector of any one of embodiments 1-18, wherein the glycoprotein derived from an envelope glycoprotein F of a virus of the Paramyxoviridae family comprises a cytoplasmic tail deletion of the F protein.
20. The pseudotyped viral particle or viral vector of any one of embodiments 1-18, wherein the glycoprotein derived from an envelope glycoprotein F of a virus of the Paramyxoviridae family comprises a cytoplasmic tail deletion of the F protein, wherein the full length F protein comprises the sequence of SEQ ID NO: 3.
21. The pseudotyped viral particle or viral vector of any one of embodiments 1-18, wherein the glycoprotein derived from an envelope glycoprotein F of a virus of the Paramyxoviridae family comprises is a Nipah F protein (“NiV-F protein”).
22. The pseudotyped viral particle or viral vector of embodiment 21, wherein the NiV-F protein has a cytoplasmic tail truncation comprising the deletion of amino acid residues 526-546 of SEQ ID NO: 3.
23. The pseudotyped viral particle or viral vector of embodiment 21, wherein the NiV-F protein comprises a cytoplasmic tail lacking amino residues 525-544 of SEQ ID NO: 3.
24. The pseudotyped viral particle or viral vector of embodiment 21, wherein the cytoplasmic tail of the NiV-F protein is truncated after amino acid residue 524 of SEQ ID NO: 3.
25. The pseudotyped viral particle or viral vector of embodiment 21, wherein the cytoplasmic tail of the NiV-F protein is truncated after amino acid residue 525 of SEQ ID NO: 3.
26. The pseudotyped viral particle or viral vector of embodiment 21, wherein the cytoplasmic tail of the NiV-F protein is truncated after amino acid residue 526 of SEQ ID NO: 3.
27. The pseudotyped viral particle or viral vector of any one of embodiments 21-26, wherein the NiV-F protein comprises a substitution of glutamine for asparagine at an amino acid position that corresponds to position 99 of SEQ ID NO: 3.
28. The pseudotyped viral particle or viral vector of embodiment 21, wherein the NiV-F protein comprises an amino acid sequence having at least 90%, 91%, 92%, 93%, 94%, 95%, 96%, 97%, 98%, 99%, or 100% identity to SEQ ID NO: 4.
29. The pseudotyped viral particle or viral vector of embodiment 1, wherein:
   the targeting polypeptide comprises a heterologous targeting moiety linked to a NiV-G;
   the truncated protein comprises a mutated NiV-G protein that does not comprise a targeting moiety; and a
   the glycoprotein derived from an envelope glycoprotein F of a virus of the Paramyxoviridae family is NiV-F protein,
   wherein the targeting polypeptide and the truncated protein comprise the same or different NiV-G protein.
30. The pseudotyped viral particle or viral vector of embodiment 29, wherein the targeting polypeptide and the truncated protein comprise the same NiV-G protein.
31. The pseudotyped viral particle or viral vector of embodiment 29 or 30, wherein the targeting polypeptide and the truncated protein, each independently, comprise a NiV glycoprotein G protein (“NiV-G”) comprising a N-terminal deletion.
32. The pseudotyped viral particle or viral vector of embodiment 29 or 30, wherein the targeting polypeptide and the truncated protein, each independently, comprise a NiV glycoprotein G protein (“NiV-G protein”) comprising a deletion of the cytoplasmic portion.
33. The pseudotyped viral particle or viral vector of embodiment 29 or 30, wherein the targeting polypeptide and the truncated protein, each independently, comprise a NiV-G protein comprising a deletion of at least 10 contiguous amino acid residues from the cytoplasmic tail.
34. The pseudotyped viral particle or viral vector of embodiment 29 or 30, wherein the targeting polypeptide and the truncated protein, each independently, comprise a NiV-G protein comprising a deletion of at least 15 contiguous amino acid residues from the cytoplasmic tail.
35. The pseudotyped viral particle or viral vector of embodiment 29 or 30, wherein the targeting polypeptide and the truncated protein, each independently, comprise a NiV-G protein comprising a deletion of at least 20 contiguous amino acid residues from the cytoplasmic tail.
36. The pseudotyped viral particle or viral vector of embodiment 29 or 30, wherein the targeting polypeptide and the truncated protein, each independently, comprise a NiV-G protein comprising a deletion located within two amino acid residues of the N-terminus of the NiV-G protein.
37. The pseudotyped viral particle or viral vector of embodiment 29 or 30, wherein the targeting polypeptide and the truncated protein, each independently, comprise a NiV-G protein comprising a deletion comprising amino acid residues 3-7, 3-12, 3-17, 3-22, or 3-27 of SEQ ID NO: 1.
38. The pseudotyped viral particle or viral vector of embodiment 29 or 30, wherein the targeting polypeptide and the truncated protein, each independently, comprise a NiV-G protein comprising a cytoplasmic tail truncation consisting of deletion of amino acid residues 1-34 of SEQ ID NO: 1.
39. The pseudotyped viral particle or viral vector of embodiment 29 or 30, wherein the targeting polypeptide and the truncated protein, each independently, comprise a NiV-G protein comprising an amino acid sequence having at least 90%, 91%, 92%, 93%, 94%, 95%, 96%, 97%, 98%, 99%, or 100% identity to SEQ ID NO: 2.
40. The pseudotyped viral particle or viral vector of any one of embodiments 29-39, wherein the glycoprotein derived from an envelope glycoprotein F of a virus of the Paramyxoviridae family comprises is a Nipah F protein (“NiV-F protein”).
41. The pseudotyped viral particle or viral vector of any one of embodiments 29-39, wherein the NiV-F protein has a cytoplasmic tail truncation comprising the deletion of amino acid residues 526-546 of SEQ ID NO: 3.
42. The pseudotyped viral particle or viral vector of any one of embodiments 29-39, wherein the NiV-F protein comprises a cytoplasmic tail lacking amino residues 525-544 of SEQ ID NO: 3.
43. The pseudotyped viral particle or viral vector of any one of embodiments 29-39, wherein the cytoplasmic tail of the NiV-F protein is truncated after amino acid residue 524 of SEQ ID NO: 3.
44. The pseudotyped viral particle or viral vector of any one of embodiments 29-39, wherein the cytoplasmic tail of the NiV-F protein is truncated after amino acid residue 525 of SEQ ID NO: 3.
45. The pseudotyped viral particle or viral vector of any one of embodiments 29-39, wherein the cytoplasmic tail of the NiV-F protein is truncated after amino acid residue 526 of SEQ ID NO: 3.
46. The pseudotyped viral-like particle or viral vector of any one of embodiments 1-6, wherein the virus of the Paramyxoviridae family is a morbillivirus.
47. The pseudotyped viral-like particle or viral vector of any one of embodiments 1-6, wherein the glycoprotein derived from an envelope glycoprotein F of a virus of the Paramyxoviridae family is MeV-F.
48. The pseudotyped viral-like particle or viral vector of embodiment 47, wherein the MeV-F protein comprises an amino acid sequence having at least 90%, 91%, 92%, 93%, 94%, 95%, 96%, 97%, 98%, 99%, or 100% identity to SEQ ID NO: 5.
49. The pseudotyped viral-like particle or viral vector of embodiment 47, wherein the MeV-F protein comprises a truncated cytoplasmic portion.
50. The pseudotyped viral-like particle or viral vector of embodiment 49, wherein the truncated cytoplasmic portion of the F protein comprises at least 1 positively charged amino acid residue and no more than 9 consecutive amino acid residues as counted from the N-terminal end of the cytoplasmic portion of the F protein.
51. The pseudotyped viral-like particle or viral vector of embodiment 49, wherein the truncated cytoplasmic portion of the measles F protein comprises:

(SEQ ID NO: 5)
MGLKVNVSAIFMAVLLTLQTPTGQIHWGNLSKIGVVGIGSASYKV
MTRSSHQSLVIKLMPNITLLNNCTRVEIAEYRRLLRTVLEPIRDA
LNAMTQNIRPVQSVASSRRHKRFAGVVLAGAALGVATAAQITAGI
ALHQSMLNSQAIDNLRASLETTNQAIEAIRQAGQEMILAVQGVQD
YINNELIPSMNQLSCDLIGQKLGLKLLRYYTEILSLFGPSLRDPI
SAEISIQALSYALGGDINKVLEKLGYSGGDLLGILESRGIKARIT
HVDTESYLIVLSIAYPTLSEIKGVIVHRLEGVSYNIGSQEWYTTV
PKYVATQGYLISNFDESSCIFMPEGTVCSQNALYPMSPLLQECLR
GSTKSCARTLVSGSFGNRFILSQGNLIANCASILCKCYTTGTIIN
QDPDKILTYIAADHCPVVEVNGVTIQVGSRRYPDAVYLHRIDLGP
PILLERLDVGINLGNAIAKLEDAKELLESSDQILRSMKGLSSTCI
VYILIAVCLGGLIGIPALICCCRGR.

52. The pseudotyped viral-like particle or viral vector of any one of embodiments 47-51, wherein the targeting polypeptide and the truncated protein, each independently, comprise a MeV-H protein.
53. The pseudotyped viral-like particle or viral vector of embodiment 52, wherein the MeV-H protein comprises a truncated MeV-H protein.
54. The pseudotyped viral-like particle or viral vector of embodiment 53, wherein the truncated MeV-H protein comprises HcΔ14, HcΔ15, HcΔ16, HcΔ17, HcΔ18, HcΔ19, HcΔ20, HcΔ21+A or HcΔ24+4A as provided herein and as described in U.S. Pat. No. 10,415,057, which is hereby incorporated by reference.
55. The pseudotyped viral-like particle or viral vector of embodiment 53, wherein the truncated MeV-H protein comprises

(SEQ ID NO: 6)
MGSRIVINREHLMIDRPYVLLAVLFVMFLSLIGLLAIAGIRLHRA
AIYTAEIHKSLSINLDVINSIEHQVKDVLIPLFKIIGDEVGLRTP
QRFTDLVKFISDKIKFLNPDREYDERDLTWCINPPERIKLDYDQY
CADVAAEELMNALVNSTLLETRTTNQFLAVSKGNCSGPTTIRGQF
SNMSLSLLDLYLGRGYNVSSIVTMTSQGMYGGTYLVEKPNLSSKR
SELSQLSMYRVFEVGVIRNPGLGAPVFHMTNYLEQPVSNDLSNCM
VALGELKLAALCHGEDSITIPYQGSGKGVSFQLVKLGVWKSPTDM
QSWVPLSTDDPVIDRLYLSSHRGVIADNQAKWAVPTTRIDDKLRM
ETCFQQACKGKIQALCENPEWAPLKDNRIPSYGVLSVDLSLIVEL
KIKIASGFGPLITHGSGMDLYKSNHNNVYWLTIPPMKNLALGVIN
TLEWIPRFKVSPALFNVPIKEAGEDCHAPTYLPAEVDGDVKLSSN
LVILPGQDLQYVLATYDTSAVEHAVVYYVYSPSRLSSYFYPFRLP
IKGVPIELQVECFTWDQKLWCRHFCVLADSESGGHITHSGMVGMG
VSCTVTREDGTNRRGG.

56. A pseudotyped viral-like particle or viral vector comprises a plurality of polypeptides comprising the general amino acid formula of:

[PG]-[RR]-Linker-[TM];

[EG]; and

[GF], wherein
PG is an envelope glycoprotein G or H sequence of a virus of the Paramyxoviridae family, such as, but not limited to, as provided herein;
RR is an optional restriction site sequence, such as, but not limited to, as provided herein;
Linker is a peptide linker, such as, but not limited to, as provided herein;
TM is a targeting moiety, such as, but not limited to, as provided herein;
EG is an envelope glycoprotein G or H of a virus of the Paramyxoviridae family, such as, but not limited to, as provided herein; and
GF is an envelope glycoprotein F sequence of a virus of the Paramyxoviridae family, such as, but not limited to, as provided herein.
57. The pseudotyped viral particle or viral vector of embodiment 56, wherein the PG comprises a NiV glycoprotein G protein (“NiV-G”) comprising a N-terminal deletion.
58. The pseudotyped viral particle or viral vector of embodiment 56, wherein the PG comprises a NiV glycoprotein G protein (“NiV-G protein”) comprising a deletion of the cytoplasmic portion.
59. The pseudotyped viral particle or viral vector of embodiment 58, wherein the NiV-G protein comprises a deletion of at least 10 contiguous amino acid residues from the cytoplasmic tail.
60. The pseudotyped viral particle or viral vector of embodiment 58, wherein the NiV-G protein comprises a deletion of at least 15 contiguous amino acid residues from the cytoplasmic tail.
61. The pseudotyped viral particle or viral vector of embodiment 58, wherein the NiV-G protein comprises a deletion of at least 20 contiguous amino acid residues from the cytoplasmic tail.
62. The pseudotyped viral particle or viral vector of embodiment 58, wherein the NiV-G protein comprises a deletion comprising amino acid residues 3-7, 3-12, 3-17, 3-22, or 3-27 of SEQ ID NO: 1.
63. The pseudotyped viral particle or viral vector of embodiment 58, wherein the NiV-G protein comprises a cytoplasmic tail truncation consisting of deletion of amino acid residues 1-34 of SEQ ID NO: 1.
64. The pseudotyped viral particle or viral vector of embodiment 56, wherein the EG sequence comprises an amino acid sequence having at least 90%, 91%, 92%, 93%, 94%, 95%, 96%, 97%, 98%, 99%, or 100% identity to SEQ ID NO: 2.
65. The pseudotyped viral particle or viral vector of any one of embodiments 56-64, wherein the GF sequence comprises a sequence derived from an envelope glycoprotein F of a virus of the Paramyxoviridae family comprises a cytoplasmic tail deletion of the F protein.
66. The pseudotyped viral particle or viral vector of any one of embodiments 56-65, wherein the GF sequence comprises a sequence derived from an envelope glycoprotein F of a virus of the Paramyxoviridae family comprises a cytoplasmic tail deletion of the F protein, wherein the full length F protein comprises the sequence of SEQ ID NO: 3.
67. The pseudotyped viral particle or viral vector of any one of embodiments 59-66, wherein the GF sequence comprises a sequence derived from an envelope glycoprotein F of a virus of the Paramyxoviridae family comprises is a Nipah F protein (“NiV-F protein”).
68. The pseudotyped viral particle or viral vector of embodiment 67, wherein the NiV-F protein has a cytoplasmic tail truncation comprising the deletion of amino acid residues 526-546 of SEQ ID NO: 3.
69. The pseudotyped viral particle or viral vector of embodiment 67, wherein the NiV-F protein comprises a cytoplasmic tail lacking amino residues 525-544 of SEQ ID NO: 3.
70. The pseudotyped viral particle or viral vector of embodiment 67, wherein the cytoplasmic tail of the NiV-F protein is truncated after amino acid residue 524 of SEQ ID NO: 3.
71. The pseudotyped viral particle or viral vector of embodiment 67, wherein the cytoplasmic tail of the NiV-F protein is truncated after amino acid residue 525 of SEQ ID NO: 3.
72. The pseudotyped viral particle or viral vector of embodiment 67, wherein the cytoplasmic tail of the NiV-F protein is truncated after amino acid residue 526 of SEQ ID NO: 3.
73. The pseudotyped viral particle or viral vector of any one of embodiments 67-72, wherein the NiV-F protein comprises a substitution of glutamine for asparagine at an amino acid position that corresponds to position 99 of SEQ ID NO: 3.
74. The pseudotyped viral particle or viral vector of embodiment 67, wherein the NiV-F protein comprises an amino acid sequence having at least 90%, 91%, 92%, 93%, 94%, 95%, 96%, 97%, 98%, 99%, or 100% identity to SEQ ID NO: 4.
75. The pseudotyped viral-like particle or viral vector of any one of embodiments 56-74, wherein the GF sequence is a sequence derived from an envelope glycoprotein F of a virus of the Paramyxoviridae family is MeV-F.
76. The pseudotyped viral-like particle or viral vector of embodiment 75, wherein the MeV-F protein comprises an amino acid sequence having at least 90%, 91%, 92%, 93%, 94%, 95%, 96%, 97%, 98%, 99%, or 100% identity to SEQ ID NO: 5.
77. The pseudotyped viral-like particle or viral vector of embodiment 75, wherein the MeV-F protein comprises a truncated cytoplasmic portion.
78. The pseudotyped viral-like particle or viral vector of embodiment 77, wherein the truncated cytoplasmic portion of the F protein comprises at least 1 positively charged amino acid residue and no more than 9 consecutive amino acid residues as counted from the N-terminal end of the cytoplasmic portion of the F protein.
79. The pseudotyped viral-like particle or viral vector of embodiment 78, wherein the truncated cytoplasmic portion of the measles F protein comprises SEQ ID NO: 5.
80. The pseudotyped viral-like particle or viral vector of any one of embodiments 56-79, wherein the PG sequence comprises a MeV-H protein.
81. The pseudotyped viral-like particle or viral vector of embodiment 80, wherein the MeV-H protein comprises a truncated MeV-H protein.
82. The pseudotyped viral-like particle or viral vector of embodiment 80, wherein the truncated MeV-H protein comprises HcΔ14, HcΔ15, HcΔ16, HcΔ17, HcΔ18, HcΔ19, HcΔ20, HcΔ21+A or HcΔ24+4A as provided herein and as described in U.S. Pat. No. 10,415,057, which is hereby incorporated by reference.
83. The pseudotyped viral-like particle or viral vector of embodiment 80, wherein the truncated MeV-H protein comprises SEQ ID NO: 6.
84. The pseudotyped viral-like particle or viral vector of any one of embodiments 56-83, wherein the RR sequence is absent.
85. The pseudotyped viral-like particle or viral vector of any one of embodiments 56-84, wherein the RR sequence comprises the amino acid sequence of DI, or is random.
86. The pseudotyped viral-like particle or viral vector of any one of embodiments 56-85, wherein the Linker sequence is a peptide linker.
87. The pseudotyped viral-like particle or viral vector of any one of embodiments 56-85, wherein the Linker sequence is absent.
88. The pseudotyped viral-like particle or viral vector of any one of embodiments 1-87, wherein the targeting moiety is an antibody, a scFv antibody, an antigen binding domain, an ankyrin repeat (e.g., DARPIN), a VHH domain antibody, a nanobody, single domain antibody, a FN3 domain, or any combination thereof.
89. The pseudotyped viral-like particle or viral vector of embodiment 88, wherein the targeting moiety is a scFv.
90. The pseudotyped viral-like particle or viral vector of embodiment 88, wherein the targeting moiety is an ankyrin repeat.
91. The pseudotyped viral-like particle or viral vector of any one of embodiments 1-90, wherein the targeting moiety binds to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsgl); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-la); Carbonic anhydrase IX (CA1X); Carcinoembryonic antigen (CEA); CCCTC-Binding Factor (Zinc Finger Protein)-Like (BORIS or Brother of the Regulator of Imprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECLI); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLVI-Tax; Human papilloma virus E6 (HPV E6); Human papilloma virus E7 (HPV E7); Human Telomerase reverse transcriptase (hTERT); IgE; IL13Ra2; ILl 1Ra; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-1 (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAP1; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member 1A (XAGE1).
92. The pseudotyped viral-like particle or viral vector of any one of embodiments 1-91, wherein the particle or vector further comprises a heterologous nucleic acid molecule encoding a cargo of interest.
93. The pseudotyped viral-like particle or viral vector of embodiment 92, wherein the cargo of interest is a siRNA, shRNA, a protein or portion thereof, a chimeric antigen receptor, an antibody, or a tumor antigen or a portion thereof.
94. The pseudotyped viral-like particle or viral vector of embodiment 92, wherein the heterologous nucleic acid molecule encoding the cargo of interest encodes for a chimeric antigen receptor (CAR).
95. The pseudotyped viral-like particle or viral vector of any one of embodiments 1-94, wherein the viral particle is a recombinant lentivirus.
96. A pharmaceutical composition comprising the pseudotyped viral-like particle or viral vector of any one of embodiments 1-95.
97. The pharmaceutical composition of embodiment 96, further comprising a pharmaceutically acceptable carrier.
98. A method of delivering a cargo of interest to a cell, the method comprising contacting the cell with the pseudotyped viral-like particle or viral vector of any one of embodiments 1-95, or a pharmaceutical composition comprising the same.
99. A method of delivering a cargo of interest to a cell in a subject, the method comprising administering to the subject the pseudotyped viral-like particle or viral vector of any one of embodiments 1-95, or a pharmaceutical composition comprising the same.
100. A method for of delivering a chimeric antigen receptor to a target cell in a subject, the method comprising administering to the subject the pseudotyped viral-like particle or viral vector of any one of embodiments 1-95, or a pharmaceutical composition comprising the same, wherein the pseudotyped viral-like particle or viral vector comprises a heterologous nucleic acid molecule encoding the chimeric antigen receptor.
101. The method of embodiment 100, wherein the targeting moiety binds to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsgl); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-la); Carbonic anhydrase IX (CA1X); Carcinoembryonic antigen (CEA); CCCTC-Binding Factor (Zinc Finger Protein)-Like (BORIS or Brother of the Regulator of lmprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECLI); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLVl-Tax; Human papilloma virus E6 (HPV E6); Human papilloma virus E7 (HPV E7); Human Telomerase reverse transcriptase (hTERT); IgE; IL13Ra2; ILl 1Ra; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-1 (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAPI; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member 1A (XAGE1).
102. The method of embodiment 100, wherein the targeting moiety binds to CD7 or CD8.
103. The method of embodiment 100, wherein the target cell is an immune cell, T cell, B cell, NK cell, dendritic cell, neutrophils, macrophages, cancer cell, CD3+ T cell, CD4+ T cell, CD7+ T cell, CD8+ T cell, CD19+ B cell, CD19+ cancer cell, CD20+ B cell, CD30+ lung epithelial cell, CD34+ haematopoietic stem cell, CD105+ endothelial cell, CD105+ haematopoietic stem cell, CD117+ haematopoietic stem cell, CD133+ cancer cell, EpCAM+ cancer cell, GluA2+ neuron, GluA4+ neuron, Haematopoietic stem cell, Hepatocyte, Her2/Neu+ cancer cell, NKG2D+ natural killer cell, SLC1A3+ astrocyte, or SLC7A10+ adipocyte.
104. A method of treating cancer in a subject, the method comprising administering to the subject the pseudotyped viral-like particle or viral vector of any one of embodiments 1-95, or a pharmaceutical composition comprising the same, wherein the pseudotyped viral-like particle or viral vector comprises a heterologous nucleic acid molecule encoding the chimeric antigen receptor.
105. A nucleic acid molecule encoding a targeting polypeptide comprising a heterologous targeting moiety linked to a protein derived from an envelope glycoprotein G or H of a virus of the Paramyxoviridae family;
   a truncated protein derived from an envelope glycoprotein G or H of a virus of the Paramyxoviridae family, wherein the truncated protein does not comprise a targeting moiety;
   a glycoprotein derived from an envelope glycoprotein F of a virus of the Paramyxoviridae family,
   wherein the targeting polypeptide and the truncated protein comprise the same or different envelope glycoproteins.
106. A plurality of nucleic acid molecules encoding a targeting polypeptide comprising a heterologous targeting moiety linked to a protein derived from an envelope glycoprotein G or H of a virus of the Paramyxoviridae family;
   a truncated protein derived from an envelope glycoprotein G or H of a virus of the Paramyxoviridae family, wherein the truncated protein does not comprise a targeting moiety;
   a glycoprotein derived from an envelope glycoprotein F of a virus of the Paramyxoviridae family,
   wherein the targeting polypeptide and the truncated protein comprise the same or different envelope glycoproteins.
107. A method of making a pseudotyped viral-like particle or viral vector of any one of embodiments 1-95, the method comprising transfecting or transducing a packaging cell line with the nucleic acid molecule of embodiments 105 or 106 under conditions sufficient to produce the pseudotyped viral-like particle or viral vector of any one of embodiments 1-95.
108. The method of embodiment 107, further comprising isolating the pseudotyped viral-like particle or viral vector.
109. A kit comprising a pharmaceutical composition comprising the pseudotyped viral-like particle or viral vector of any one of embodiments 1-95.

The following examples are illustrative, but not limiting, of the compounds, compositions and methods described herein. Other suitable modifications and adaptations known to those skilled in the art are within the scope of the following embodiments.

### EXAMPLES

Example 1: A pseudo-typed lentivirus with a targeting moiety polypeptide NiV-G is created via transient transfection of producer cells (e.g., 293T cells) using standard transfection reagents (e.g., lipofectamine 3000). Plasmids amounting to 7.5 μg of Rev, 10 μg×HIV gag/pol, 15 μg of transgene (e.g., model antigen (GFP) or CAR), 8 μg of NiV-F protein, 4 μg of NiV-G protein encoding an extracellular targeting moiety (e.g., scFv, Darpin, VHH), and 1 μg of a non-targeted NiV-G protein are mixed and transfection is conducted as per manufacturer's recommendations. Briefly, 90 μL of P3000 reagent is mixed with the plasmids and then subsequently mixed with 90 μL of Lipofectamine 3000 reagent which is applied to the producer cells. Two days later, viruses are collected and concentrated via standard methods (e.g., centrifugation, chromatography, filtration) and resuspended in an appropriate media.

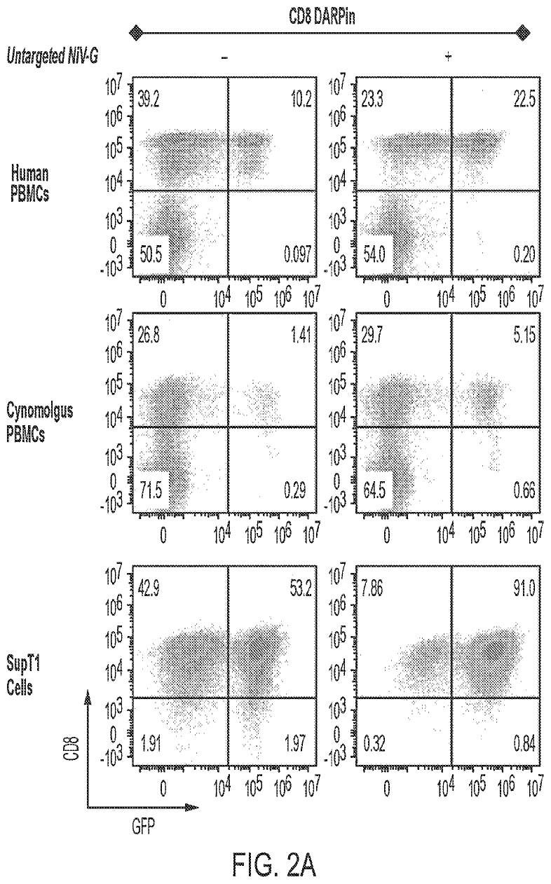

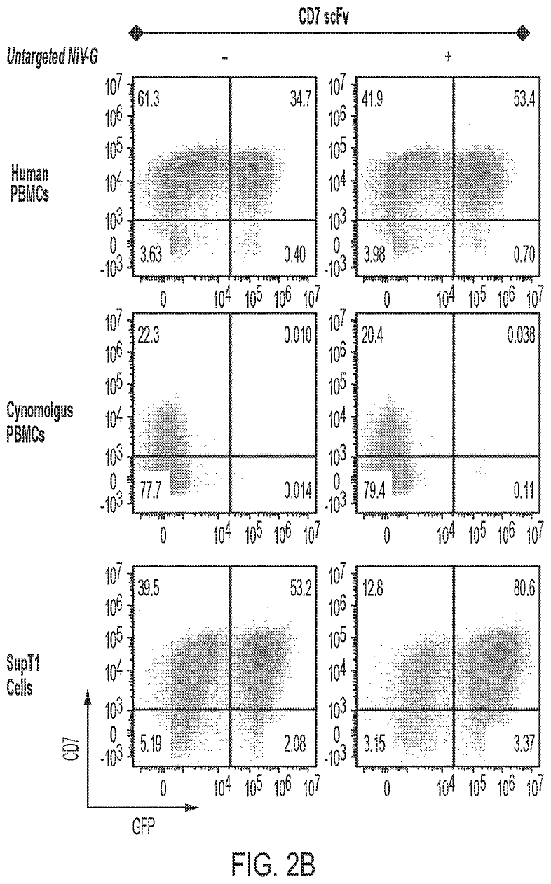

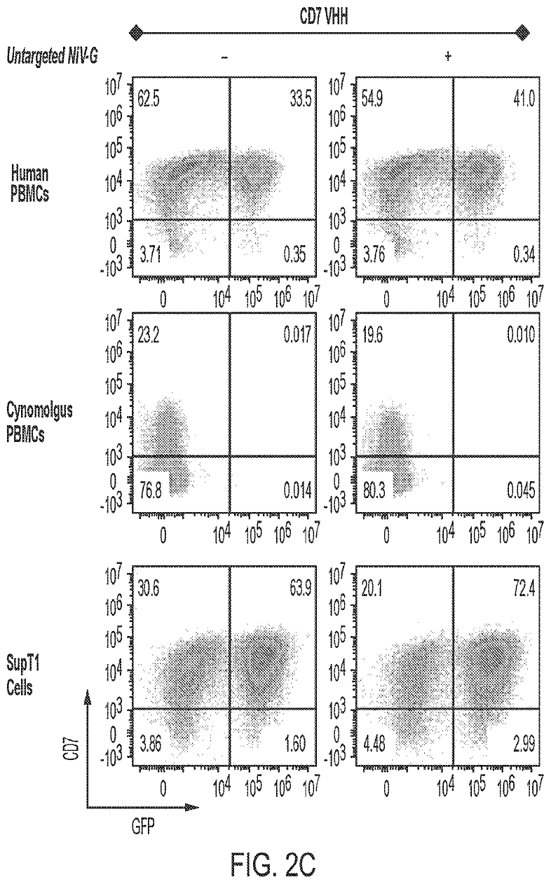

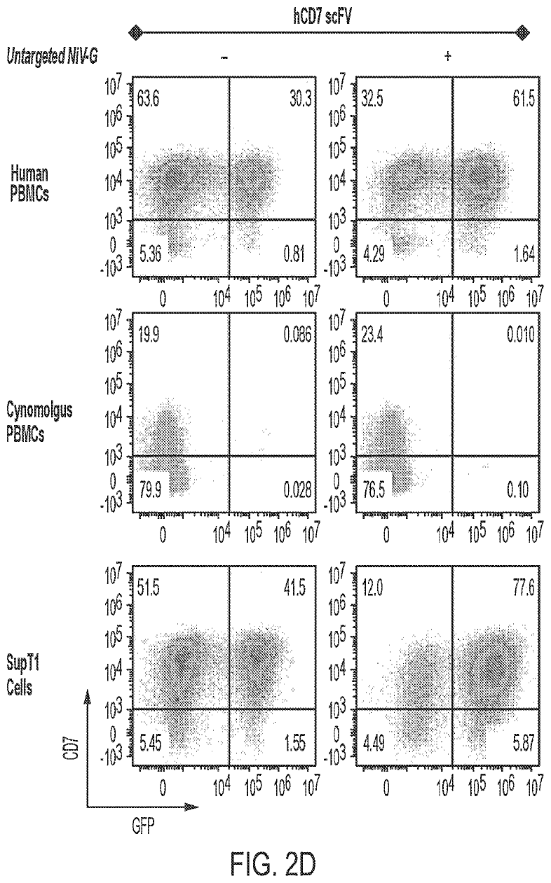

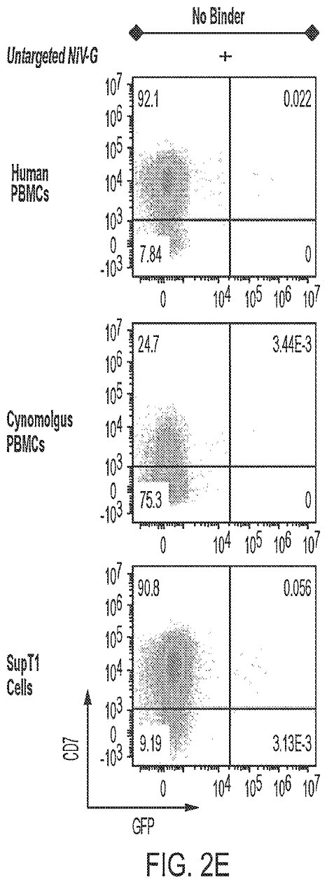

Example 2: A pseudo-typed lentivirus with a targeting moiety polypeptide NiV-G fusion protein and a non-targeted Niv-G protein enhances transduction as compared to virus without the untargeted Niv-G protein. Following lentiviral preparation as outlined in example 1, target cells (e.g., human PBMCs, cynomolgus PBMCs, or model T cells (Supt1)) are transduced by incubating viruses with the cells. 7 days after transduction, the expression of the transgene (e.g., GFP) is determined via flow cytometry. These data can be seen in FIG. 2A-E. Values for viral vectors containing a targeting moiety alone or a targeting moiety and a non-targeting moiety reveal that the addition of a non-targeting moiety increases transduction. The table below shows the percent of cells that are positive for expression of both the transgene and the extracellular targeting moiety produced by each set of conditions.

| | CD8 Darpin | | CD7 scFv | | CD7 VHH | | humanized CD7 scFv | | |
|---|---|---|---|---|---|---|---|---|---|
| Model | | Targeting + | | Targeting + | | Targeting + | | Targeting + | Non- |
| Transgene | Targeting | non- | Targeting | non- | Targeting | non- | Targeting | non- | targeted |
| Transduction | alone | targeted G | alone | targeted G | alone | targeted G | alone | targeted G | alone |
| Human PBMCs | 10.2 | 22.5 | 34.7 | 53.4 | 33.5 | 41 | 30.3 | 61.5 | <1 |
| Cynomolgus | 1.41 | 5.15 | <1 | <1 | <1 | <1 | <1 | <1 | <1 |
| PBMCs | | | | | | | | | |
| Supt1 Cells | 53.2 | 91 | 53.2 | 80.6 | 63.9 | 72.4 | 41.5 | 77.6 | <1 |

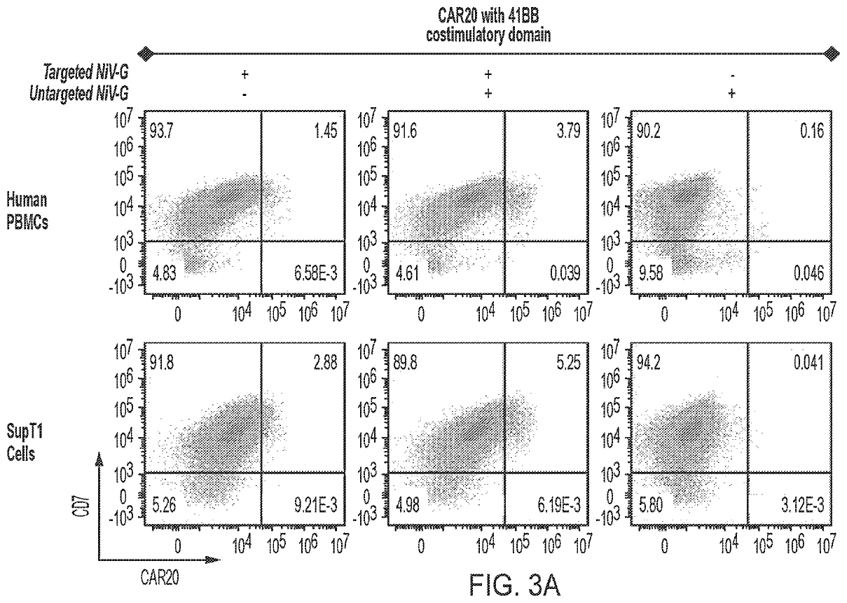

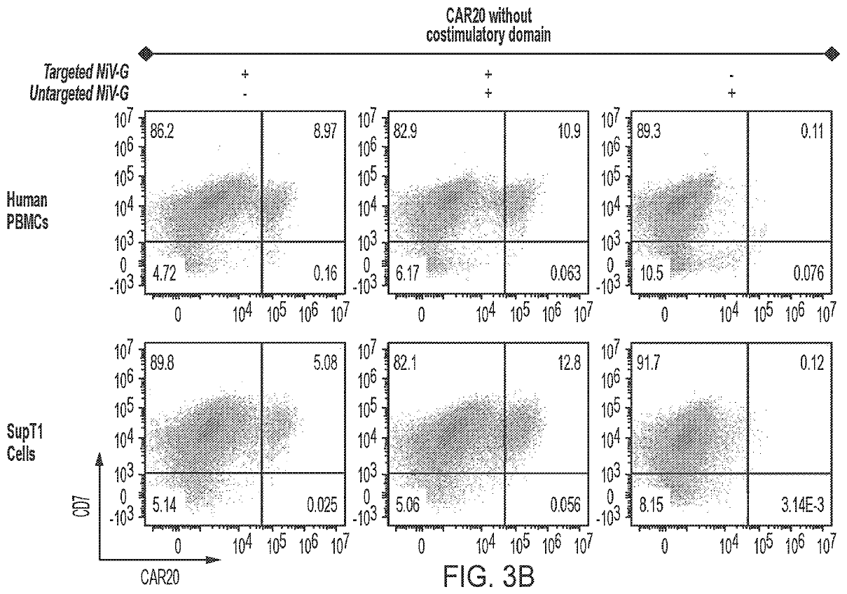

Example 3: A pseudo-typed lentivirus expressing a chimeric antigen receptor along with a targeting moiety polypeptide Niv-G protein and a non-targeted Niv-G protein enhances transduction as compared to virus without the untargeted Niv-G protein. Following lentiviral preparation as outlined in example 1, target cells (e.g., human PBMCs or model T cells (Supt1)) are transduced by incubating viruses with the cells. 7 days after transduction, the expression of the transgene (e.g., CAR20 with a 41BB costimulatory domain or CAR20 without a costimulatory domain) is determined via flow cytometry. These data can be seen in FIG. 3A-B. Values for viral vectors containing a targeting moiety alone or a targeting moiety and a non-targeting moiety reveal that the addition of a non-targeting moiety increases transduction. The table below shows the percent of cells that are positive for expression of both the transgene and the extracellular targeting moiety produced by each set of conditions.

| | | | | CAR20 without | | |
|---|---|---|---|---|---|---|
| CAR20 | CAR20 with 41BB | | | costimulatory domain | | |
| Transduction | | Targeting + | Non- | | Targeting + | Non- |
| with a | Targeting | non- | targeted | Targeting | non- | targeted |
| CD7 scFv | alone | targeted G | alone | alone | targeted G | alone |
| Human PBMCs | 1.45 | 3.79 | <1 | 8.97 | 10.9 | <1 |
| Supt1 Cells | 2.88 | 5.25 | <1 | 5.08 | 12.8 | <1 |

The Examples demonstrate that molecules provided herein can be used to transduce cells with greater efficiency than viral-like particles without the compositions provided for herein. Such effects were surprising and could not have been predicted.

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

What is claimed:

1. An pseudotyped viral-like particle or viral vector comprising:
a targeting polypeptide comprising a heterologous targeting moiety linked to a protein derived from an envelope glycoprotein G or H of a virus of the Paramyxoviridae family;
a truncated protein derived from an envelope glycoprotein G or H of a virus of the Paramyxoviridae family, wherein the truncated protein does not comprise a targeting moiety;
a glycoprotein derived from an envelope glycoprotein F of a virus of the Paramyxoviridae family,
wherein the targeting polypeptide and the truncated protein comprise the same or different envelope glycoproteins.

2. The pseudotyped viral-like particle or viral vector of claim 1, wherein the viral-like particle is a retroviral-like particle or retroviral vector.

3. The pseudotyped viral-like particle or viral vector of claim 2, wherein the retroviral-like particle is a lentivirus based viral particle or viral vector.

4. The pseudotyped viral-like particle or viral vector of any one of claims 1-3, wherein the envelope glycoprotein G or H of a virus of the Paramyxoviridae family of the targeting polypeptide is a modified envelope glycoprotein G or H of a virus of the Paramyxoviridae family.

5. The pseudotyped viral-like particle or viral vector of claim 4, wherein the modified envelope glycoprotein G or H of a virus of the Paramyxoviridae family comprises at least one mutation.

6. The pseudotyped viral-like particle or viral vector of claim 4, wherein the modified envelope glycoprotein G or H of a virus does not bind to its native target protein.

7. The pseudotyped viral-like particle or viral vector of any one of claims 1-6, wherein the virus of the Paramyxoviridae family is a henipavirus.

8. The pseudotyped viral-like particle or viral vector of claim 7, wherein the virus of the Henipavirus genus is Nipah virus, Cedar virus, or Hendra virus.

9. The pseudotyped viral-like particle or viral vector of claim 7, wherein the virus of the Henipavirus genus is Nipah virus (“NiV”).

10. The pseudotyped viral particle or viral vector of claim 9, wherein the targeting polypeptide comprises a NiV glycoprotein G protein (“NiV-G”) comprising a N-terminal deletion.

11. The pseudotyped viral particle or viral vector of claim 9, wherein the targeting polypeptide comprises a NiV glycoprotein G protein (“NiV-G protein”) comprising a deletion of the cytoplasmic portion.

12. The pseudotyped viral particle or viral vector of claim 9, wherein the NiV-G protein comprises a deletion of at least 10 contiguous amino acid residues from the cytoplasmic tail.

13. The pseudotyped viral particle or viral vector of claim 9, wherein the NiV-G protein comprises a deletion of at least 15 contiguous amino acid residues from the cytoplasmic tail.

14. The pseudotyped viral particle or viral vector of claim 9, wherein the NiV-G protein comprises a deletion of at least 20 contiguous amino acid residues from the cytoplasmic tail.

15. The pseudotyped viral particle or viral vector of claim 9, wherein the NiV-G protein comprises a deletion located within two amino acid residues of the N-terminus of the NiV-G protein.

16. The pseudotyped viral particle or viral vector of claim 9, wherein the NiV-G protein comprises a deletion comprising amino acid residues 3-7, 3-12, 3-17, 3-22, or 3-27 of SEQ ID NO: 1.

17. The pseudotyped viral particle or viral vector of claim 9, wherein the NiV-G protein comprises a cytoplasmic tail truncation consisting of deletion of amino acid residues 1-34 of SEQ ID NO: 1.

18. The pseudotyped viral particle or viral vector of claim 8, wherein the second NiV-G protein comprises an amino acid sequence having at least 90%, 91%, 92%, 93%, 94%, 95%, 96%, 97%, 98%, 99%, or 100% identity to SEQ ID NO: 2.

19. The pseudotyped viral particle or viral vector of any one of claims 1-18, wherein the glycoprotein derived from an envelope glycoprotein F of a virus of the Paramyxoviridae family comprises a cytoplasmic tail deletion of the F protein.

20. The pseudotyped viral particle or viral vector of any one of claims 1-18, wherein the glycoprotein derived from an envelope glycoprotein F of a virus of the Paramyxoviridae family comprises a cytoplasmic tail deletion of the F protein, wherein the full length F protein comprises the sequence of SEQ ID NO: 3.

21. The pseudotyped viral particle or viral vector of any one of claims 1-18, wherein the glycoprotein derived from an envelope glycoprotein F of a virus of the Paramyxoviridae family comprises is a Nipah F protein (“NiV-F protein”).

22. The pseudotyped viral particle or viral vector of claim 21, wherein the NiV-F protein has a cytoplasmic tail truncation comprising the deletion of amino acid residues 526-546 of SEQ ID NO: 3.

23. The pseudotyped viral particle or viral vector of claim 21, wherein the NiV-F protein comprises a cytoplasmic tail lacking amino residues 525-544 of SEQ ID NO: 3.

24. The pseudotyped viral particle or viral vector of claim 21, wherein the cytoplasmic tail of the NiV-F protein is truncated after amino acid residue 524 of SEQ ID NO: 3.

25. The pseudotyped viral particle or viral vector of claim 21, wherein the cytoplasmic tail of the NiV-F protein is truncated after amino acid residue 525 of SEQ ID NO: 3.

26. The pseudotyped viral particle or viral vector of claim 21, wherein the cytoplasmic tail of the NiV-F protein is truncated after amino acid residue 526 of SEQ ID NO: 3.

27. The pseudotyped viral particle or viral vector of any one of claims 21-26, wherein the NiV-F protein comprises a substitution of glutamine for asparagine at an amino acid position that corresponds to position 99 of SEQ ID NO: 3.

28. The pseudotyped viral particle or viral vector of claim 21, wherein the NiV-F protein comprises an amino acid sequence having at least 90%, 91%, 92%, 93%, 94%, 95%, 96%, 97%, 98%, 99%, or 100% identity to SEQ ID NO: 4.

29. The pseudotyped viral particle or viral vector of claim 1, wherein:
the targeting polypeptide comprises a heterologous targeting moiety linked to a NiV-G;
the truncated protein comprises a mutated NiV-G protein that does not comprise a targeting moiety; and a
the glycoprotein derived from an envelope glycoprotein F of a virus of the Paramyxoviridae family is NiV-F protein,
wherein the targeting polypeptide and the truncated protein comprise the same or different NiV-G protein.

30. The pseudotyped viral particle or viral vector of claim 29, wherein the targeting polypeptide and the truncated protein comprise the same NiV-G protein.

31. The pseudotyped viral particle or viral vector of claim 29 or 30, wherein the targeting polypeptide and the truncated protein, each independently, comprise a NiV glycoprotein G protein (“NiV-G”) comprising a N-terminal deletion.

32. The pseudotyped viral particle or viral vector of claim 29 or 30, wherein the targeting polypeptide and the truncated protein, each independently, comprise a NiV glycoprotein G protein (“NiV-G protein”) comprising a deletion of the cytoplasmic portion.

33. The pseudotyped viral particle or viral vector of claim 29 or 30, wherein the targeting polypeptide and the truncated protein, each independently, comprise a NiV-G protein comprising a deletion of at least 10 contiguous amino acid residues from the cytoplasmic tail.

34. The pseudotyped viral particle or viral vector of claim 29 or 30, wherein the targeting polypeptide and the truncated protein, each independently, comprise a NiV-G protein comprising a deletion of at least 15 contiguous amino acid residues from the cytoplasmic tail.

35. The pseudotyped viral particle or viral vector of claim 29 or 30, wherein the targeting polypeptide and the truncated protein, each independently, comprise a NiV-G protein comprising a deletion of at least 20 contiguous amino acid residues from the cytoplasmic tail.

36. The pseudotyped viral particle or viral vector of claim 29 or 30, wherein the targeting polypeptide and the truncated protein, each independently, comprise a NiV-G protein comprising a deletion located within two amino acid residues of the N-terminus of the NiV-G protein.

37. The pseudotyped viral particle or viral vector of claim 29 or 30, wherein the targeting polypeptide and the truncated protein, each independently, comprise a NiV-G protein comprising a deletion comprising amino acid residues 3-7, 3-12, 3-17, 3-22, or 3-27 of SEQ ID NO: 1.

38. The pseudotyped viral particle or viral vector of claim 29 or 30, wherein the targeting polypeptide and the truncated protein, each independently, comprise a NiV-G protein comprising a cytoplasmic tail truncation consisting of deletion of amino acid residues 1-34 of SEQ ID NO: 1.

39. The pseudotyped viral particle or viral vector of claim 29 or 30, wherein the targeting polypeptide and the truncated protein, each independently, comprise a NiV-G protein comprising an amino acid sequence having at least 90%, 91%, 92%, 93%, 94%, 95%, 96%, 97%, 98%, 99%, or 100% identity to SEQ ID NO: 2.

40. The pseudotyped viral particle or viral vector of any one of claims 29-39, wherein the glycoprotein derived from an envelope glycoprotein F of a virus of the Paramyxoviridae family comprises is a Nipah F protein (“NiV-F protein”).

41. The pseudotyped viral particle or viral vector of any one of claims 29-39, wherein the NiV-F protein has a cytoplasmic tail truncation comprising the deletion of amino acid residues 526-546 of SEQ ID NO: 3.

42. The pseudotyped viral particle or viral vector of any one of claims 29-39, wherein the NiV-F protein comprises a cytoplasmic tail lacking amino residues 525-544 of SEQ ID NO: 3.

43. The pseudotyped viral particle or viral vector of any one of claims 29-39, wherein the cytoplasmic tail of the NiV-F protein is truncated after amino acid residue 524 of SEQ ID NO: 3.

44. The pseudotyped viral particle or viral vector of any one of claims 29-39, wherein the cytoplasmic tail of the NiV-F protein is truncated after amino acid residue 525 of SEQ ID NO: 3.

45. The pseudotyped viral particle or viral vector of any one of claims 29-39, wherein the cytoplasmic tail of the NiV-F protein is truncated after amino acid residue 526 of SEQ ID NO: 3.

46. The pseudotyped viral-like particle or viral vector of any one of claims 1-6, wherein the virus of the Paramyxoviridae family is a morbillivirus.

47. The pseudotyped viral-like particle or viral vector of any one of claims 1-6, wherein the glycoprotein derived from an envelope glycoprotein F of a virus of the Paramyxoviridae family is MeV-F.

48. The pseudotyped viral-like particle or viral vector of claim 47, wherein the MeV-F protein comprises an amino acid sequence having at least 90%, 91%, 92%, 93%, 94%, 95%, 96%, 97%, 98%, 99%, or 100% identity to SEQ ID NO: 5.

49. The pseudotyped viral-like particle or viral vector of claim 47, wherein the MeV-F protein comprises a truncated cytoplasmic portion.

50. The pseudotyped viral-like particle or viral vector of claim 49, wherein the truncated cytoplasmic portion of the F protein comprises at least 1 positively charged amino acid residue and no more than 9 consecutive amino acid residues as counted from the N-terminal end of the cytoplasmic portion of the F protein.

51. The pseudotyped viral-like particle or viral vector of claim 49, wherein the truncated cytoplasmic portion of the measles F protein comprises:
(SEQ ID NO: 5)
MGLKVNVSAIFMAVLLTLQTPTGQIHWGNLSKIGVVGIGSASYKV
MTRSSHQSLVIKLMPNITLLNNCTRVEIAEYRRLLRTVLEPIRDA
LNAMTQNIRPVQSVASSRRHKRFAGVVLAGAALGVATAAQITAGI
ALHQSMLNSQAIDNLRASLETINQAIEAIRQAGQEMILAVQGVQD
YINNELIPSMNQLSCDLIGQKLGLKLLRYYTEILSLFGPSLRDPI
SAEISIQALSYALGGDINKVLEKLGYSGGDLLGILESRGIKARIT
HVDTESYLIVLSIAYPTLSEIKGVIVHRLEGVSYNIGSQEWYTTV
PKYVATQGYLISNFDESSCTFMPEGTVCSQNALYPMSPLLQECLR
GSTKSCARTLVSGSFGNRFILSQGNLIANCASILCKCYTTGTIIN
QDPDKILTYIAADHCPVVEVNGVTIQVGSRRYPDAVYLHRIDLGP
PILLERLDVGTNLGNAIAKLEDAKELLESSDQILRSMKGLSSTCI
VYILIAVCLGGLIGIPALICCCRGR.

52. The pseudotyped viral-like particle or viral vector of any one of claims 47-51, wherein the targeting polypeptide and the truncated protein, each independently, comprise a MeV-H protein.

53. The pseudotyped viral-like particle or viral vector of claim 52, wherein the MeV-H protein comprises a truncated MeV-H protein.

54. The pseudotyped viral-like particle or viral vector of claim 53, wherein the truncated MeV-H protein comprises HcΔ14, HcΔ15, HcΔ16, HcΔ17, HcΔ18, HcΔ19, HcΔ20, HcΔ21+A or HcΔ24+4A.

55. The pseudotyped viral-like particle or viral vector of claim 53, wherein the truncated MeV-H protein comprises
(SEQ ID NO: 6)
MGSRIVINREHLMIDRPYVLLAVLFVMFLSLIGLLAIAGIRLHRA
AIYTAEIHKSLSTNLDVINSIEHQVKDVLIPLFKIIGDEVGLRTP
QRFTDLVKFISDKIKFLNPDREYDERDLTWCINPPERIKLDYDQY
CADVAAEELMNALVNSTLLETRITNQFLAVSKGNCSGPTTIRGQF
SNMSLSLLDLYLGRGYNVSSIVTMISQGMYGGTYLVEKPNLSSKR
SELSQLSMYRVFEVGVIRNPGLGAPVFHMTNYLEQPVSNDLSNCM
VALGELKLAALCHGEDSITIPYQGSGKGVSFQLVKLGVWKSPTDM
QSWVPLSTDDPVIDRLYLSSHRGVIADNQAKWAVPTTRTDDKLRM
ETCFQQACKGKIQALCENPEWAPLKDNRIPSYGVLSVDLSLIVEL
KIKIASGFGPLITHGSGMDLYKSNHNNVYWLTIPPMKNLALGVIN
TLEWIPRFKVSPALFNVPIKEAGEDCHAPTYLPAEVDGDVKLSSN
LVILPGQDLQYVLATYDTSAVEHAVVYYVYSPSRLSSYFYPFRLP
IKGVPIELQVECFTWDQKLWCRHFCVLADSESGGHITHSGMVGMG
VSCTVTREDGTNRRGG.

56. A pseudotyped viral-like particle or viral vector comprises a plurality of polypeptides comprising the general amino acid formula of:
[PG]-[RR]-Linker-[TM];
[EG]; and
[GF], wherein
PG is an envelope glycoprotein G or H sequence of a virus of the Paramyxoviridae family;
RR is an optional restriction site sequence;
Linker is a peptide linker;
TM is a targeting moiety;
EG is an envelope glycoprotein G or H of a virus of the Paramyxoviridae family; and
GF is an envelope glycoprotein F sequence of a virus of the Paramyxoviridae family.

57. The pseudotyped viral particle or viral vector of claim 56, wherein the PG comprises a NiV glycoprotein G protein (“NiV-G”) comprising a N-terminal deletion.

58. The pseudotyped viral particle or viral vector of claim 56, wherein the PG comprises a NiV glycoprotein G protein (“NiV-G protein”) comprising a deletion of the cytoplasmic portion.

59. The pseudotyped viral particle or viral vector of claim 58, wherein the NiV-G protein comprises a deletion of at least 10 contiguous amino acid residues from the cytoplasmic tail.

60. The pseudotyped viral particle or viral vector of claim 58, wherein the NiV-G protein comprises a deletion of at least 15 contiguous amino acid residues from the cytoplasmic tail.

61. The pseudotyped viral particle or viral vector of claim 58, wherein the NiV-G protein comprises a deletion of at least 20 contiguous amino acid residues from the cytoplasmic tail.

62. The pseudotyped viral particle or viral vector of claim 58, wherein the NiV-G protein comprises a deletion comprising amino acid residues 3-7, 3-12, 3-17, 3-22, or 3-27 of SEQ ID NO: 1.

63. The pseudotyped viral particle or viral vector of claim 58, wherein the NiV-G protein comprises a cytoplasmic tail truncation consisting of deletion of amino acid residues 1-34 of SEQ ID NO: 1.

64. The pseudotyped viral particle or viral vector of claim 56, wherein the EG sequence comprises an amino acid sequence having at least 90%, 91%, 92%, 93%, 94%, 95%, 96%, 97%, 98%, 99%, or 100% identity to SEQ ID NO: 2.

65. The pseudotyped viral particle or viral vector of any one of claims 56-64, wherein the GF sequence comprises a sequence derived from an envelope glycoprotein F of a virus of the Paramyxoviridae family comprises a cytoplasmic tail deletion of the F protein.

66. The pseudotyped viral particle or viral vector of any one of claims 56-65, wherein the GF sequence comprises a sequence derived from an envelope glycoprotein F of a virus of the Paramyxoviridae family comprises a cytoplasmic tail deletion of the F protein, wherein the full length F protein comprises the sequence of SEQ ID NO: 3.

67. The pseudotyped viral particle or viral vector of any one of claims 59-66, wherein the GF sequence comprises a sequence derived from an envelope glycoprotein F of a virus of the Paramyxoviridae family comprises is a Nipah F protein (“NiV-F protein”).

68. The pseudotyped viral particle or viral vector of claim 67, wherein the NiV-F protein has a cytoplasmic tail truncation comprising the deletion of amino acid residues 526-546 of SEQ ID NO: 3.

69. The pseudotyped viral particle or viral vector of claim 67, wherein the NiV-F protein comprises a cytoplasmic tail lacking amino residues 525-544 of SEQ ID NO: 3.

70. The pseudotyped viral particle or viral vector of claim 67, wherein the cytoplasmic tail of the NiV-F protein is truncated after amino acid residue 524 of SEQ ID NO: 3.

71. The pseudotyped viral particle or viral vector of claim 67, wherein the cytoplasmic tail of the NiV-F protein is truncated after amino acid residue 525 of SEQ ID NO: 3.

72. The pseudotyped viral particle or viral vector of claim 67, wherein the cytoplasmic tail of the NiV-F protein is truncated after amino acid residue 526 of SEQ ID NO: 3.

73. The pseudotyped viral particle or viral vector of any one of claims 67-72, wherein the NiV-F protein comprises a substitution of glutamine for asparagine at an amino acid position that corresponds to position 99 of SEQ ID NO: 3.

74. The pseudotyped viral particle or viral vector of claim 67, wherein the NiV-F protein comprises an amino acid sequence having at least 90%, 91%, 92%, 93%, 94%, 95%, 96%, 97%, 98%, 99%, or 100% identity to SEQ ID NO: 4.

75. The pseudotyped viral-like particle or viral vector of any one of claims 56-74, wherein the GF sequence is a sequence derived from an envelope glycoprotein F of a virus of the Paramyxoviridae family is MeV-F.

76. The pseudotyped viral-like particle or viral vector of claim 75, wherein the MeV-F protein comprises an amino acid sequence having at least 90%, 91%, 92%, 93%, 94%, 95%, 96%, 97%, 98%, 99%, or 100% identity to SEQ ID NO: 5.

77. The pseudotyped viral-like particle or viral vector of claim 75, wherein the MeV-F protein comprises a truncated cytoplasmic portion.

78. The pseudotyped viral-like particle or viral vector of claim 77, wherein the truncated cytoplasmic portion of the F protein comprises at least 1 positively charged amino acid residue and no more than 9 consecutive amino acid residues as counted from the N-terminal end of the cytoplasmic portion of the F protein.

79. The pseudotyped viral-like particle or viral vector of claim 78, wherein the truncated cytoplasmic portion of the measles F protein comprises SEQ ID NO: 5.

80. The pseudotyped viral-like particle or viral vector of any one of claims 56-79, wherein the PG sequence comprises a MeV-H protein.

81. The pseudotyped viral-like particle or viral vector of claim 80, wherein the MeV-H protein comprises a truncated MeV-H protein.

82. The pseudotyped viral-like particle or viral vector of claim 80, wherein the truncated MeV-H protein comprises HcΔ14, HcΔ15, HcΔ16, HcΔ17, HcΔ18, HcΔ19, HcΔ20, HcΔ21+A or HcΔ24+4A.

83. The pseudotyped viral-like particle or viral vector of claim 80, wherein the truncated MeV-H protein comprises SEQ ID NO: 6.

84. The pseudotyped viral-like particle or viral vector of any one of claims 56-83, wherein the RR sequence is absent.

85. The pseudotyped viral-like particle or viral vector of any one of claims 56-84, wherein the RR sequence comprises the amino acid sequence of DI, or is random.

86. The pseudotyped viral-like particle or viral vector of any one of claims 56-85, wherein the Linker sequence is a peptide linker.

87. The pseudotyped viral-like particle or viral vector of any one of claims 56-85, wherein the Linker sequence is absent.

88. The pseudotyped viral-like particle or viral vector of any one of claims 1-87, wherein the targeting moiety is an antibody, a scFv antibody, an antigen binding domain, an ankyrin repeat (e.g., DARPIN), a VHH domain antibody, a nanobody, single domain antibody, a FN3 domain, or any combination thereof.

89. The pseudotyped viral-like particle or viral vector of claim 88, wherein the targeting moiety is a scFv.

90. The pseudotyped viral-like particle or viral vector of claim 88, wherein the targeting moiety is an ankyrin repeat.

91. The pseudotyped viral-like particle or viral vector of any one of claims 1-90, wherein the targeting moiety binds to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsgl); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-la); Carbonic anhydrase IX (CA1X); Carcinoembryonic antigen (CEA); CCCTC-Binding Factor (Zinc Finger Protein)-Like (BORIS or Brother of the Regulator of lmprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECLI); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLVl-Tax; Human papilloma virus E6 (HPV E6); Human papilloma virus E7 (HPV E7); Human Telomerase reverse transcriptase (hTERT); IgE; IL13Ra2; IL11Ra; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-1 (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAP1; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member 1A (XAGE1).

92. The pseudotyped viral-like particle or viral vector of any one of claims 1-91, wherein the particle or vector further comprises a heterologous nucleic acid molecule encoding a cargo of interest.

93. The pseudotyped viral-like particle or viral vector of claim 92, wherein the cargo of interest is a siRNA, shRNA, a protein or portion thereof, a chimeric antigen receptor, an antibody, or a tumor antigen or a portion thereof.

94. The pseudotyped viral-like particle or viral vector of claim 92, wherein the heterologous nucleic acid molecule encoding the cargo of interest encodes for a chimeric antigen receptor (CAR).

95. The pseudotyped viral-like particle or viral vector of any one of claims 1-94, wherein the viral particle is a recombinant lentivirus.

96. A pharmaceutical composition comprising the pseudotyped viral-like particle or viral vector of any one of claims 1-95.

97. The pharmaceutical composition of claim 96, further comprising a pharmaceutically acceptable carrier.

98. A method of delivering a cargo of interest to a cell, the method comprising contacting the cell with the pseudotyped viral-like particle or viral vector of any one of claims 1-95, or a pharmaceutical composition comprising the same.

99. A method of delivering a cargo of interest to a cell in a subject, the method comprising administering to the subject the pseudotyped viral-like particle or viral vector of any one of claims 1-95, or a pharmaceutical composition comprising the same.

100. A method for of delivering a chimeric antigen receptor to a target cell in a subject, the method comprising administering to the subject the pseudotyped viral-like particle or viral vector of any one of claims 1-95, or a pharmaceutical composition comprising the same, wherein the pseudotyped viral-like particle or viral vector comprises a heterologous nucleic acid molecule encoding the chimeric antigen receptor.

101. The method of claim 100, wherein the targeting moiety binds to CD7 or CD8.

102. The method of embodiment 100, wherein the target cell is an immune cell, T cell, B cell, NK cell, dendritic cell, neutrophils, macrophages, cancer cell, CD3+ T cell, CD4+ T cell, CD7+ T cell, CD8+ T cell, CD19+ B cell, CD19+ cancer cell, CD20+ B cell, CD30+ lung epithelial cell, CD34+ haematopoietic stem cell, CD105+ endothelial cell, CD105+ haematopoietic stem cell, CD117+ haematopoietic stem cell, CD133+ cancer cell, EpCAM+ cancer cell, GluA2+ neuron, GluA4+ neuron, Haematopoietic stem cell, Hepatocyte, Her2/Neu+ cancer cell, NKG2D+ natural killer cell, SLC1A3+ astrocyte, and SLC7A10+ adipocyte.

103. A method of treating cancer in a subject, the method comprising administering to the subject the pseudotyped viral-like particle or viral vector of any one of claims 1-95, or a pharmaceutical composition comprising the same, wherein the pseudotyped viral-like particle or viral vector comprises a heterologous nucleic acid molecule encoding the chimeric antigen receptor.

104. A nucleic acid molecule encoding a targeting polypeptide comprising a heterologous targeting moiety linked to a protein derived from an envelope glycoprotein G or H of a virus of the Paramyxoviridae family;
a truncated protein derived from an envelope glycoprotein G or H of a virus of the Paramyxoviridae family, wherein the truncated protein does not comprise a targeting moiety;
a glycoprotein derived from an envelope glycoprotein F of a virus of the Paramyxoviridae family,
wherein the targeting polypeptide and the truncated protein comprise the same or different envelope glycoproteins.

105. A plurality of nucleic acid molecules encoding a targeting polypeptide comprising a heterologous targeting moiety linked to a protein derived from an envelope glycoprotein G or H of a virus of the Paramyxoviridae family;
a truncated protein derived from an envelope glycoprotein G or H of a virus of the Paramyxoviridae family, wherein the truncated protein does not comprise a targeting moiety;
a glycoprotein derived from an envelope glycoprotein F of a virus of the Paramyxoviridae family,
wherein the targeting polypeptide and the truncated protein comprise the same or different envelope glycoproteins.

106. A method of making a pseudotyped viral-like particle or viral vector of any one of claims 1-95, the method comprising transfecting or transducing a packaging cell line with the nucleic acid molecule of claim 104 or 105 under conditions sufficient to produce the pseudotyped viral-like particle or viral vector of any one of claims 1-95.

107. The method of claim 106, further comprising isolating the pseudotyped viral-like particle or viral vector.

108. A kit comprising a pharmaceutical composition comprising the pseudotyped viral-like particle or viral vector of any one of claims 1-95.
